(19) United States
(12) Patent Application Publication
(10) Pub. No.: US 2023/0219862 A1
(43) Pub. Date: Jul. 13, 2023

(54) SYSTEM AND METHOD OF ISOLATION, SELECTION, AND USE OF INDIGENOUS MICROBES FOR CARBON CAPTURE AND INCREASING THE WATER HOLDING CAPACITY IN AGRICULTURAL SOILS

(71) Applicant: MYLAND COMPANY, LLC, Phoenix, AZ (US)

(72) Inventors: Dane N. Hague, Phoenix, AZ (US); Andrew D. Ayers, Phoenix, AZ (US); Matthew J. Olson, Phoenix, AZ (US)

(21) Appl. No.: 17/973,481

(22) Filed: Oct. 25, 2022

Related U.S. Application Data
(60) Provisional application No. 63/271,511, filed on Oct. 25, 2021.

(51) Int. Cl. C05F 11/08; C12M 1/00

(52) U.S. Cl. CPC C05F 11/08; C12M 21/02

(57) ABSTRACT

In some embodiments, the systems and methods described herein are directed to using microbes such as algae to capture carbon in multiple stages. In some embodiments, during an initial algae growth phase, the system is configured to enable algae to capture carbon dioxide. In some embodiments, a method includes using indigenous algae and/or other microbes from the same environment where the algae and/or other microbes will eventually be distributed. In some embodiments, the initial algae growth phase is a first carbon capture phase. In some embodiments, as the algae grows the carbon dioxide is consumed by the algae while oxygen is released. In some embodiments, once the growth of the algae reaches a maximum capacity of the system, the algae must be expelled from the system to make room for new algae growth which in turn allows for further carbon removal from the atmosphere.

### BACKGROUND

The types of microbes in soil are often unique to the environment and geographical location. Algae have the ability to adapt to their environment. For instance, algae found in soil in the Southwestern deserts have adapted to elevated temperatures, alkaline pH levels, and periods of desiccation, while algae in northern climates have adapted to much lower temperatures, freeze-thaw cycles, higher soil moisture levels, and more acidic soil pH levels, etc. Most native soil algae are mixotrophic (able to utilize sugars and other organic molecules as a food source) as sunlight does not penetrate the soil.

Indigenous algae fill a niche in the field ecosystem. Within the soil ecosystem, a symbiosis with other organisms has developed resulting in a biochemical environment where compounds produced by the indigenous algae may augment the growth of other desirable microbes and depress the growth of undesirable or non-beneficial organisms. For example, algae are known to produce biochemicals such as amino acids, hormones, peptides, and fatty acids that augment the growth of beneficial microorganisms. These beneficial biochemicals also directly help the crop plants. The beneficial microorganisms produce biochemicals that the algae and crop can utilize to grow (e.g., sugars and vitamins) resulting in continued algal and crop growth. At the same time, algae may produce compounds that are antibacterial, antifungal, algicidal, and/or antiprotozoal which prevent the growth of unwanted microbes in the soil.

Currently, there is no technology that utilizes algae as both a system for capturing carbon and a system to promote plant growth. Algae used in water purification systems, for example, simply discard the algae as a manufacturing waste, ignoring the potential secondary benefits of this diverse plant. In addition, using indigenous algae species in any agricultural system as a carbon capture source such that the discarded algae can be used to promote growth in a specific environment has never been contemplated.

Therefore, there is a need in the art for a system that uses microbes such as algae to combat the rise of greenhouse gases by capturing carbon in both a growth and decay cycle.

### SUMMARY

In some embodiments, the systems and methods described herein are directed to a novel way of capturing carbon. In some embodiments, a method step includes preparing one or more microbe-containing samples from at least one location of a current or planned plant growth area. In some embodiments, a method step includes preparing at least one cultured sample by culturing microbes from the sample. In some embodiments, a method step includes selecting at least one target species of microbe from the at least one cultured sample. In some embodiments, a method step includes propagating the at least one selected target species of microbe to increase the concentration of the at least one target species of microbe in the at least one cultured sample. In some embodiments, a method step includes delivering at least a portion of the at least one target species microbe to at least a portion of the at least one location, wherein at least a portion of the at least one target species of microbe being delivered comprises at least one live microbe.

In some embodiments, the at least one selected target species of microbe includes an algal species. In some embodiments, the at least one selected target species of microbe includes a bacterial species. In some embodiments, the at least one selected target species of microbe includes a fungal species. In some embodiments, the at least one selected target species of microbe includes a mixotrophic capable algal species. In some embodiments, the at least one selected target species of microbe is selected based at least in part on its ability to produce biomass.

In some embodiments, the propagating of the at least one selected target species of microbe is performed in a bioreactor vessel. In some embodiments, the bioreactor vessel is an algae production vessel. In some embodiments, the bioreactor vessel is a photobioreactor. In some embodiments, the bioreactor is located onsite at the at least one location of a current or planned plant growth area. In some embodiments, the bioreactor is located offsite from the at least one location of a current or planned plant growth area.

In some embodiments, a result of the delivering at least a portion of the at least one target species of microbe to at least a portion of the at least one location includes an increase in a concentration of the at least one target species in the at least one location. In some embodiments, the increase in concentration comprises an increase in concentration beyond a naturally occurring concentration of the at least one target species of microbe of the portion of the at least one location.

In some embodiments, the increase in concentration leads to a reduction in soil salinity. In some embodiments, the increase in concentration leads to an increase in soil organic carbon. In some embodiments, the increase in concentration leads to an increase in soil organic matter (SOM). In some embodiments, the increase in concentration leads to an increase in the bioavailability of macro and micronutrients. In some embodiments, the increase in concentration leads to improved soil permeability and water retention. In some embodiments, the increase in concentration leads to improved crop yields in at least a portion of the at least one location. In some embodiments, the increase in concentration leads to improved nutrient value and/or overall quality of one or more crops grown in at least a portion of the at least one location.

In some embodiments, the increase in concentration leads to a reduction in fertilizer usage in at least a portion of the at least one location. In some embodiments, the increase in concentration leads to a reduction in pollution to waterways from chemical runoff from at least a portion of the at least one location. In some embodiments, the current or planned plant growth area includes at least one of an agricultural growth area, a farm, a garden, a greenhouse, a forest, a desert, reclaimed land, and a golf course. In some embodiments, the one or more microbe-containing samples comprises at least one of water, soil, or water-soil mixture.

### DRAWINGS DESCRIPTION

FIG. 1 shows TOC change at soil depths of 0-12″ for a first soil location according to some embodiments.

FIG. 2 illustrates the approximate 38% increase in total organic carbon (TOC) in the soil according to some embodiments.

FIG. 3 shows a high range of bulk density according to some embodiments.

FIG. 4 shows a low range of bulk density increase for the first location according to some embodiments.

FIG. 5 shows bulk density provided by the NRCS Web Soil Survey according to some embodiments.

FIG. 6 shows TOC increase at soil depths of 0-6″, 6-12″, 12-24″, and 24-36″ at a second soil location according to some embodiments.

FIG. 7 illustrates the % change in TOC for the second soil location according to some embodiments.

FIG. 8 depicts the change in the high range of bulk density according to some embodiments.

FIG. 9 depicts the change in the low range of bulk density according to some embodiments.

FIG. 10 shows bulk density provided by the NRCS Web Soil Survey according to some embodiments.

FIG. 11 shows TOC increase at soil depths of 0-6″ at a third soil location according to some embodiments.

FIG. 12 shows the % change in TOC according to some embodiments.

FIG. 13 depicts the change in the high range of bulk density according to some embodiments.

FIG. 14 depicts the change in the low range of bulk density according to some embodiments.

FIG. 15 shows bulk density provided by the NRCS Web Soil Survey according to some embodiments.

FIG. 16 shows TOC increase at soil depths of 0-6″, 6-12″, 12-24″, and 24-36″ at a fourth soil location according to some embodiments.

FIG. 17 shows the % change in TOC according to some embodiments.

FIG. 18 depicts the change in the high range of bulk density according to some embodiments.

FIG. 19 depicts the change in the low range of bulk density according to some embodiments.

FIG. 20 shows bulk density provided by the NRCS Web Soil Survey according to some embodiments.

FIG. 21 shows TOC increase at soil depths of 0-6″ and 6-12″ at a fifth soil location according to some embodiments.

FIG. 22 shows the % change in TOC according to some embodiments.

FIG. 23 depicts the change in the high range of bulk density according to some embodiments.

FIG. 24 depicts the change in the low range of bulk density according to some embodiments.

FIG. 25 shows bulk density provided by the NRCS Web Soil Survey according to some embodiments.

FIG. 26 shows TOC increase at soil depths of 0-12″ at a sixth soil location according to some embodiments.

FIG. 27 shows the % change in TOC according to some embodiments.

FIG. 28 depicts the change in the high range of bulk density according to some embodiments.

FIG. 29 depicts the change in the low range of bulk density according to some embodiments.

FIG. 30 shows bulk density provided by the NRCS Web Soil Survey according to some embodiments.

FIG. 31 shows TOC increase at soil depths of 0-12″ at a seventh soil location according to some embodiments.

FIG. 32 shows the % change in TOC according to some embodiments.

FIG. 33 depicts the change in the high range of bulk density according to some embodiments.

FIG. 34 depicts the change in the low range of bulk density according to some embodiments.

FIG. 35 shows bulk density provided by the NRCS Web Soil Survey according to some embodiments.

FIG. 36 illustrates an amount of increased water holding capacity for the third soil location as a result of the system and methods described herein according to some embodiments.

### DETAILED DESCRIPTION

Before any embodiments of the invention are explained in detail, it is to be understood that the invention is not limited in its application to the details of isolation and propagation set forth in the following description. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown but are to be accorded the widest scope consistent with the principles and features disclosed herein. Skilled artisans will recognize the examples provided herein have many useful alternatives that fall within the scope of embodiments of the invention.

While there are many references to algae herein, such references are used solely as helpful examples, and do not limit the scope of the inventions described and claimed herein, which are directed to microbes generally as well.

In some embodiments, the systems and methods described herein are directed to a system and method for using microbes such as algae to capture carbon in multiple stages. In some embodiments, during an initial algae growth phase, the system is configured to enable algae to capture carbon dioxide. In some embodiments, a method includes using indigenous algae and/or other microbes from the same environment where the algae and/or other microbes will eventually be distributed. In some embodiments, the initial algae growth phase is a first carbon capture phase. In some embodiments, as the algae grows the carbon dioxide is consumed by the algae while oxygen is released. In some embodiments, once the growth of the algae reaches a maximum capacity of the system, the algae must be expelled from the system to make room for new algae growth which in turn allows for further carbon removal from the atmosphere. In some embodiments, a percentage of the algae growth is maintained within the system as a seed for new growth.

In some embodiments, the expulsion of algae from the system begins a second carbon capture phase. In some embodiments, a method includes expelling the algae from the system to the same environment from which it was originally sampled. In some embodiments, by constantly supplying the soil with live native algae, the relationship between the microbial community and plants will be reestablished and root exudates can again be utilized to capture even more carbon. In some embodiments, the plant provides more exudates resulting in more algae growth within the soil resulting in increased carbon capture by the new algae growth. In some embodiments, the root exudates are also utilized by many other microorganisms such as bacteria and fungi, which will also help provide benefits to plants such as providing additional nutrients, protection against non-beneficial microorganisms, and helping hold water closer and longer near the plant's root system. In some embodiments, these benefits allow the plants to grow larger which improves the carbon capture capability of the plant itself. At the same time that the algae continues to replicate and the plant's exudates feed increasing bacterial and fungal populations, portions of the soil microbial community die and become part of the ever-increasing amount of Soil Organic Matter (SOM) and the carbon locked within.

The inventors have discovered the extent of the positive effects the indigenous algae increase has on both the ground environment as a fertilizer and rejuvenator as well as the atmosphere as a carbon capturing device according to some embodiments. In some embodiments, by implementing the systems and methods described herein, land that was unsuitable for crop production now becomes fertile, and land that was fertile is now able to produce larger crops at many times the previous yield. In some embodiments, larger crops combined with multiple cuttings means more carbon removed from the atmosphere. In some embodiments, using indigenous algae to turn barren land into crop producing environments results in a carbon removal system that can be scaled exponentially. In addition, in some embodiments, the systems and methods described herein increase food resources while contributing no waste to the environment.

Plants interact with the microbiome by exuding sugars and other nutritious compounds that the microbial community can utilize as a food source. In exchange, the community can provide the plants with the nitrogen, phosphorus, potassium and other essential biochemicals they need for maximizing growth and nutritional value of their products. Unfortunately, in today's agriculture, this relationship has been destroyed from the continuous and excessive use of synthetic chemical fertilizers and poor farm practices such as tillage, which has decimated the microbial community and as a result, the plants no longer exude significant amounts of these compounds.

When soil algae die, it has been discovered that cellular biochemicals are released which can directly feed the soil biome and any crop plants growing in the soil according to some embodiments. In some embodiments, the remaining biological material accumulates in the soil as soil organic matter (SOM). Approximately 58% of SOM is in the form of carbon depending on the soil composition. In some embodiments, the other members of the soil microbiome (bacteria and fungi) grow exponentially because of the increased amount of nutrients.

In some embodiments, when algae are introduced to the soil by the systems and methods described herein, the metabolic activity in the soil increases, resulting in greater CO₂ production. This is particularly true for live algae whose exponential growth and metabolic activities continue after introduction to the soil. However, this CO₂ production is beneficial as it lowers the pH of the soil resulting in the dissolution of calcium and magnesium carbonate bonds, thereby opening the soil for greater root penetration and increased water, fertilizer, and carbon movement. This increased water movement, as a result of the increased permeability, allows the microbiome to flourish and the roots to penetrate to greater and greater depths. This results in an increase in SOM and carbon deeper into the soil. The increased SOM and carbon will go at least as deep as the plant's root system. The net effect is a larger plant biomass above and below ground and a larger microbial population so the net removal of CO₂ from the atmosphere from the first and second carbon capture phases far exceeds that produced by the metabolic activity.

The present disclosure is directed to methods of increasing water holding capacity of soil according to some embodiments. In some embodiments, it has been found that substantially constant or periodic addition of algae can result in a desirable buildup of SOM within the soil that also has the property of holding water (approximately 20,000 g/ac per 1% SOM) and nutrients which can be utilized by the plants as needed further promoting plant growth for the second carbon capture phase. In some embodiments, conventional methods of introducing carbon to the soil generally require tilling-in of organic matter (compost, various plant cuttings, manures, etc.), which can best be performed when a field is between plantings. In some embodiments, SOM provided to the growth environment by the first and second carbon capture phases described herein aids in the formation of natural iron chelates (fulvic acids-Fe), which prevents soil from being blocked by calcium and magnesium carbonates, thus avoiding chlorosis problems induced by low bioavailability of these nutrients. Accordingly, aspects of the present disclosure are directed to a method or reducing calcium and magnesium carbonates in soil according to some embodiments. Chlorosis is the reduction in the green color of plants due to a reduction in the amount of chlorophyll in the leaves brought on by a lack of bioavailable macro and micronutrients such as N, Mg, Ca, and Fe, even when these nutrients are present in the soils according to some embodiments. Some embodiments of the disclosure are directed to minimizing chlorosis.

Ion exchange capacity is a quantitative means for describing the binding of fertilizer elements to soil particles for storage and release according to some embodiments. In some embodiments, SOM ion exchange capacity (e.g., 400 to 600 meq/100 g) is 5 to 10 times higher than that of clays (e.g., 50 to 150 meq/100 g). In some embodiments, it is this capacity which allows the retention of fertilizers within the soil for use by the plants as needed. In some embodiments, as plants utilize the nitrogen (N), phosphorus (P), and Potassium (K) in the soil, the stored elements are released from the SOM as needed. In some embodiments, by combining with SOM substances using the systems and methods described herein, copper and other trace elements become less toxic and more readily available to the plants.

Some embodiments of the disclosure are directed to methods of reducing pests in soil. In some embodiments, the algae grown in the first carbon capture phase and disturbed in the second carbon capture phase plays a role in controlling agricultural pests by directly producing antibiotics and antifungal compounds, and by feeding the beneficial microbes in the soil which produce other pest fighting compounds. In some embodiments, these compounds give the plants the ability to prevent the invasion of pathogenic species. Disease and pests are also resisted due to the improved vigor of the plants. In some embodiments, this allows the plants to produce more biomass both above and below ground to improve carbon capture in the second phase. The increased carbon capture does not stop at larger sized biomass above ground as the below ground biomass captures more carbon in the form of roots.

As discussed above, in some embodiments, the systems and methods described herein use live, indigenous microalgae cells to function as a catalyst to tap and utilize all the benefits available from standard fertilizers and also to provide a natural supply of essential compounds and phytochemicals, while supporting the overall efficacy of the growing environment according to some embodiments. In some embodiments, the resulting increase in SOM traps excess standard fertilizers in a plant available form. In some embodiments, by doing this the amount of fertilizer addition needed is greatly reduced. In some embodiments, these potent attributes enabled by the systems and methods described herein work in concert to stimulate plants to grow heartier and more quickly; and to consistently produce a more abundant, higher quality and more nutrient rich end-product such as a crop. In some embodiments, the benefits from an additive of microalgae cells are available when the algae cells that are delivered to the soil are in healthy living form and in great concentration though production in the first carbon capture stage. In some embodiments, the selection and formulation of the algae additive contributes to its overall impact. In some embodiments, by using the systems and methods described herein, a microalgae additive program is simple to manage, and offers breakthrough potential in carbon capture technology as well as agricultural production. In some embodiments, the impact may be greatest in the most depleted soils such as arid soils that have significant salt and caliche buildup with minimal organic matter. In some embodiments, the system allows for the rejuvenation of depleted soil in a fraction of the time conventional regenerative agricultural methods would take, while increasing yield many times greater than conventional regenerative agricultural methods would produce.

In some embodiments, by selecting indigenous algae for propagation in the first carbon capture phase and delivery in the second carbon capture phase to an agricultural production area, the inventors have found there is a higher survival rate, a greater and faster impact on soil health and an increased carbon content in the soil in the SOM. It was discovered by the inventors that releasing a foreign (maladapted) species may result in low survivability, competition with native species, and potential disruption to the soil microbial ecosystem. Conventional methods and systems of algae production such as in water purification systems do not utilize indigenous algae. Furthermore, conventional algae production is concentrated in centralized facilities, not at or near the distribution site, thereby yielding dead algal cells, which do not confer the same benefits as live algae. Therefore, a method of constructing the first carbon capture system at or near the second carbon capture phase distribution site forms part of some of the embodiments of the system and methods described herein.

Some embodiments of the invention include methods of selecting, collecting, and growing algae for delivery to an agricultural production area. Specifically, in some embodiments, the methods focus on collecting, isolating, and/or propagating indigenous microbes, primarily algae, for mass delivery to the same biome from which the algae was collected. In some embodiments, the agricultural production area comprising the biome may be a farm field, a raised bed, a greenhouse, a golf course, degraded land, or an indoor growing facility. Some embodiments include collecting, isolating, and/or propagating, and delivering other indigenous microbes in addition to, or separately from algae. For example, some embodiments include collecting, isolating, and/or propagating, and delivering a bacterial species. Some embodiments include collecting, isolating, and/or propagating, and delivering a fungal species. In some embodiments, collecting, isolating, and/or propagating forms at least part of a first carbon capture phase. In some embodiments, delivering forms at least part of a second carbon capture phase.

In some embodiments, the algae may be delivered in the second carbon capture phase through a variety of means including, but not limited to, canal irrigation, flood irrigation, or drip irrigation, various conventional overhead spray techniques, or various conventional hydroponic cultivation techniques. In some embodiments, the effects of delivering algae to the agricultural production area may be an increase in SOM and organic carbon, improvement in soil structure, reduction in water and fertilizer utilization, increase in crop yield and the nutrient value of the product, an overall improvement in soil health, reduction in water and chemical runoff, and an increase in carbon dioxide sequestered from the air by the soil microbes.

Some embodiments include a method for a first carbon capture phase of obtaining a soil and/or water sample from an agricultural production area, and/or culturing microbes from the soil sample, and/or selecting a desirable species from the soil sample, and/or propagating the selected desirable species in greater numbers and concentration. In some embodiments, a method for a second carbon capture phase includes delivering live microbes back to the agricultural production area (e.g., such as dispersing the live microbes in solution over a soil area of a farm, or biome area). In some embodiments, the steps constitute a method for collecting, selecting, and propagating indigenous algae from an agricultural production area (e.g., such as a farm or other plant propagation facility).

Some embodiments of a first carbon capture phase include a step of collecting one or more quantities of soil from one or more locations on the agricultural production area. In some embodiments, each quantity or a total quantity of collected soil can be about 100 grams. In some other embodiments, the quantity can be less than 100 grams or more than 100 grams.

Some embodiments of a first carbon capture phase include a step of collecting one or more quantities of water from one or more locations on the agricultural production area (e.g., such as from a surface water source). In some embodiments, each quantity or a total quantity of collected water can be about 50 grams. In some other embodiments, the quantity can be less than 50 grams or more than 50 grams. In some other embodiments, at least some of the water can be collected from a sub-surface source, a run-off source, or a spring or well source.

In some embodiments, one or more of the water and/or the soil quantities can be refrigerated to 35° F. to 40° F. prior to subsequent processing locations, including, without limitation, a laboratory or facility during a first carbon capture phase.

In some embodiments, about 10 grams of soil or 10 ml of water from each sample can be added to a 100 ml culture jar containing 75 ml of AF6 (Watanabe) media during a first carbon capture phase. In some embodiments, more or less soil and/or water can be added to the culture jar. In some further embodiments, more or less AF6 (Watanabe) media can be used. In some embodiments, the soil and/or water can be incubated in the culture jar during a first carbon capture phase. In some embodiments, the incubation can occur overnight while being exposed to a 100 to 200 PAR light source. In some embodiments, the light source can contain wavelengths of 450-485 nm and 625-740 nm. In some embodiments, exposure can be 6 to 24 hours per day.

In some embodiments, at least a portion of the incubated samples can be propagated in AF6-agar-coated Petri dishes, as a non-limiting Petri dish example, during a first carbon capture phase. For example, in one non-limiting embodiment, samples can be plated-out onto 4 each 100×15 mm petri dishes with AF6 agar with 10 μ1 samples with loop sterilization in-between each streak to dilute the sample. The petri dishes can be at least partially closed (e.g., taped to 75% closed) and placed upside down in front of a 100 to 200 PAR light source for one to two weeks. In some embodiments, the light source can contain wavelengths of 450-485 nm and 625-740 nm. In some embodiments, exposure can be 6 to 24 hours per day.

In some embodiments, when isolated axenic algae colonies have grown to a specific size, the algae colonies can be harvested aseptically, and placed into a sterile test tube with sterile AF6 media during a first carbon capture phase. For example, in some embodiments, when isolated axenic algae colonies have grown to about 3 mm in diameter, the algae colonies can be harvested aseptically and placed into a sterile test tube with sterile AF6 media.

Some embodiments include incubating for one to two weeks and selecting the tubes with the highest biomass during a first carbon capture phase. In some embodiments, the incubation can occur while being exposed to a 100 to 200 PAR light source. In some embodiments, the light source can contain wavelengths of 450-485 nm and 625-740 nm. In some embodiments, exposure can be 6 to 24 hours per day. In some embodiments, temperatures can range between 70° F. and 80° F.

Some embodiments include inoculating and incubating for one to two days on a Nutrient-agar-coated Petri dishes in the absence of light and selecting the tubes which show mixotrophic capabilities (the ability to utilize organic food sources for growth). In some embodiments, temperatures can range between 70° F. and 80° F.

Some embodiments include subculturing each tube into a new tube and place the contents of the original tube into a sterile 500 ml bottle with AF6 media outfitted with sterile air injection during a first carbon capture phase. In some embodiments, the subculturing tubes can be exposed to a 100 to 200 PAR light source. In some embodiments, the light source can contain wavelengths of 450-485 nm and 625-740 nm. In some embodiments, exposure can be 6 to 24 hours per day.

Some embodiments include incubating the bottle for 3-5 days and select the bottle(s) with the fastest growth rate and highest biomass and identify with a new strain ID during a first carbon capture phase. In some embodiments, the incubation can occur while being exposed to a 100 to 200 PAR light source. In some embodiments, the light source can contain wavelengths of 450-485 nm and 625-740 nm. In some embodiments, exposure can be 6 to 24 hours per day. In some embodiments, temperatures can range between 70° F. and 80° F.

In some embodiments of the invention, the strain IDs of the incubated samples are recorded in the strain ID database with date time and location of collection along with any additional algal characteristics during a first carbon capture phase. Further, in some embodiments, new test tubes are inoculated with each newly identified strain and place in algal library for longer term preservation.

Some embodiments include an artificial selection process to improve, growth rate, maximum density, increased/decreased temperature and/or pH tolerance and other desired characteristics during a first carbon capture phase. In some embodiments, the artificial selection process can contain algae strains that are exposed to selected culture conditions. In some embodiments, algae strains that have an improved growth rate, higher maximum density, or other desired characteristics are selected over the inferior strains for future use. In some embodiments, inferior algae strains may be put through the artificial selection process to further improve the growth rate, maximum density, mixotrophic capability or other desired characteristics.

In some embodiments, one or more the steps can be performed in a laboratory or facility that is remote from the agricultural production area during a first carbon capture phase. In some embodiments of the invention, one or more the steps can be performed in a laboratory or facility that is proximate to or part of the agricultural production area. In some embodiments, all of the steps can be performed in the same location. In other embodiments, at least some of the steps can be performed in one location, and one or more other steps can be performed in another location.

The following discussion related to the figures show results of implementation of the system and methods described herein. In some embodiments, the method results in an increase in % total organic carbon (TOC) in various soil types and locations. In some embodiments, total organic carbon was measured before the addition of algae and after a certain amount of time. In some embodiments, total organic carbon was measured with dried combustion technique. In some embodiments, the soil samples are weighed in tin cups and treated with sulfurous acid to remove all forms of carbonate (inorganic carbon), leaving only organic carbon components. In some embodiments, the sample is ignited in an oxygen rich combustion chamber at approximately 1350° C. In some embodiments, an aliquot of the combustion gas is passed through an infrared absorption detector for carbon measurement.

In some embodiments, bulk density is used to quantify carbon storage. In some embodiments, tons per acre carbon storage results are estimated based on estimated bulk densities. Since TOC is reported as a percentage, to quantify actual weight stored, the bulk densities of each soils are measured. In some embodiments, the figures show tons of carbon stored calculated based on the range per soil texture and the bulk densities provided by the Web Soil Survey. In some embodiments, bulk density, one-third bar, includes the oven dry weight of the soil material less than 2 mm in size per unit volume of soil at water tension of ⅓ bar, expressed in grams per cubic centimeter. In some embodiments, bulk density is used to compute linear extensibility, shrink-swell potential, available water capacity, total pore space, and other soil properties. In some embodiments, the moist bulk density of a soil indicates the pore space available for water and roots. In some embodiments, moist bulk density is influenced by texture, kind of clay, content of organic matter, and/or soil structure.

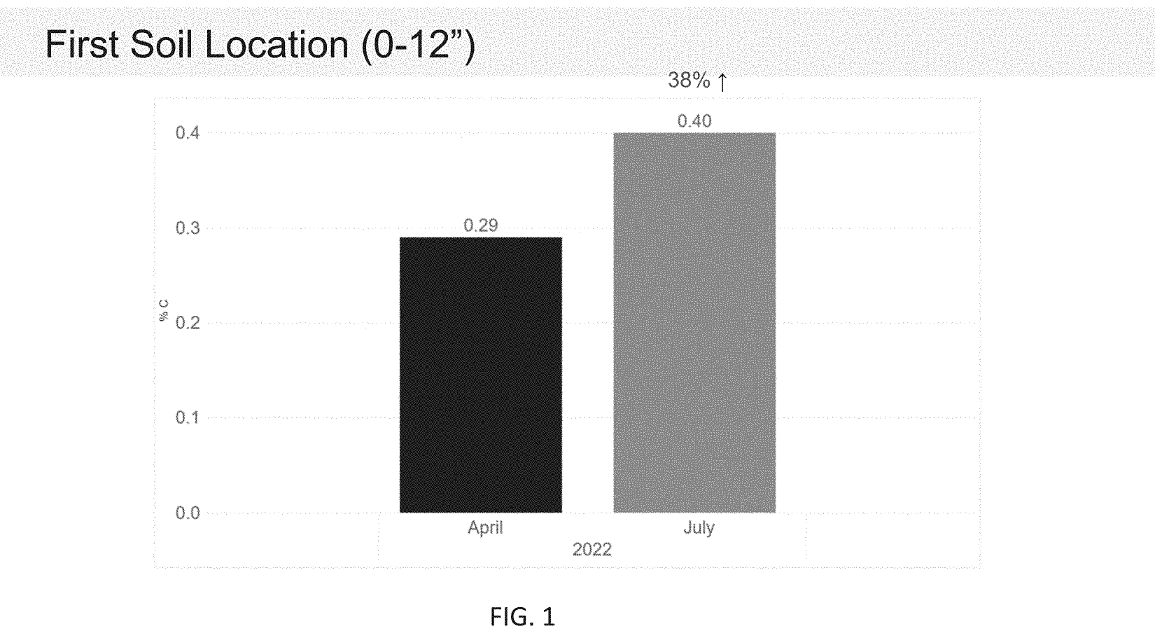

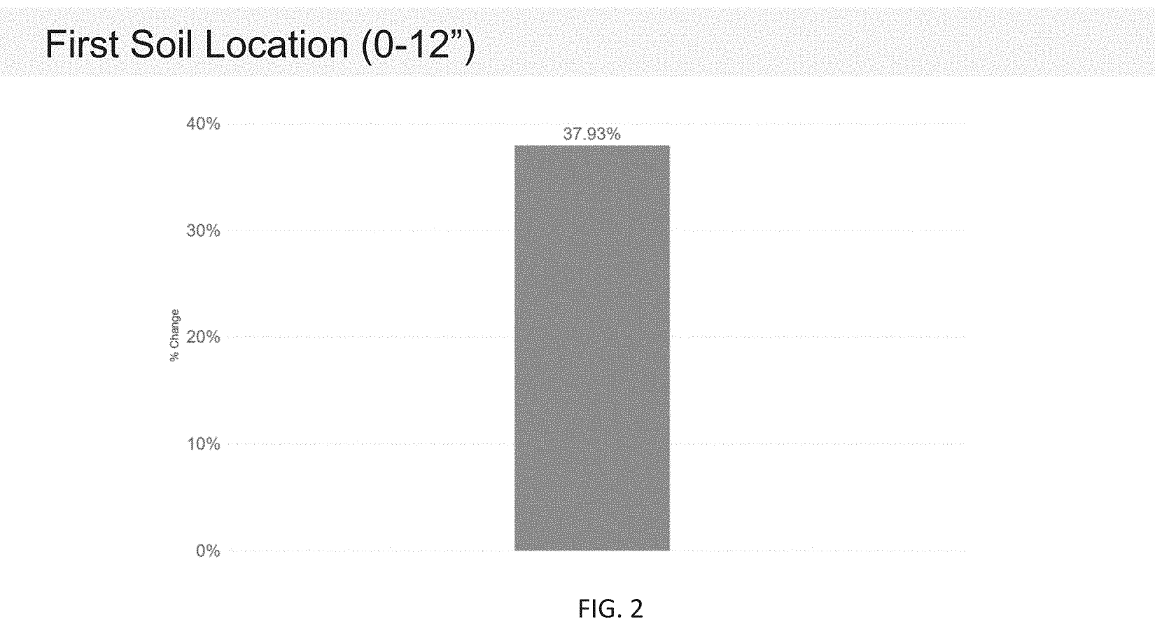

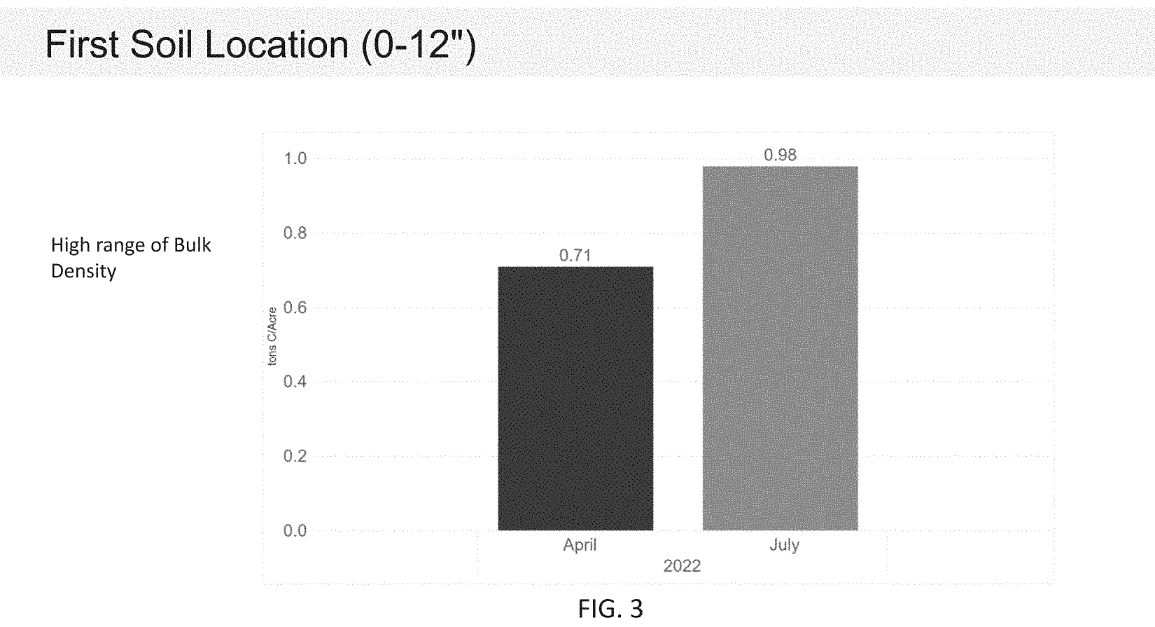

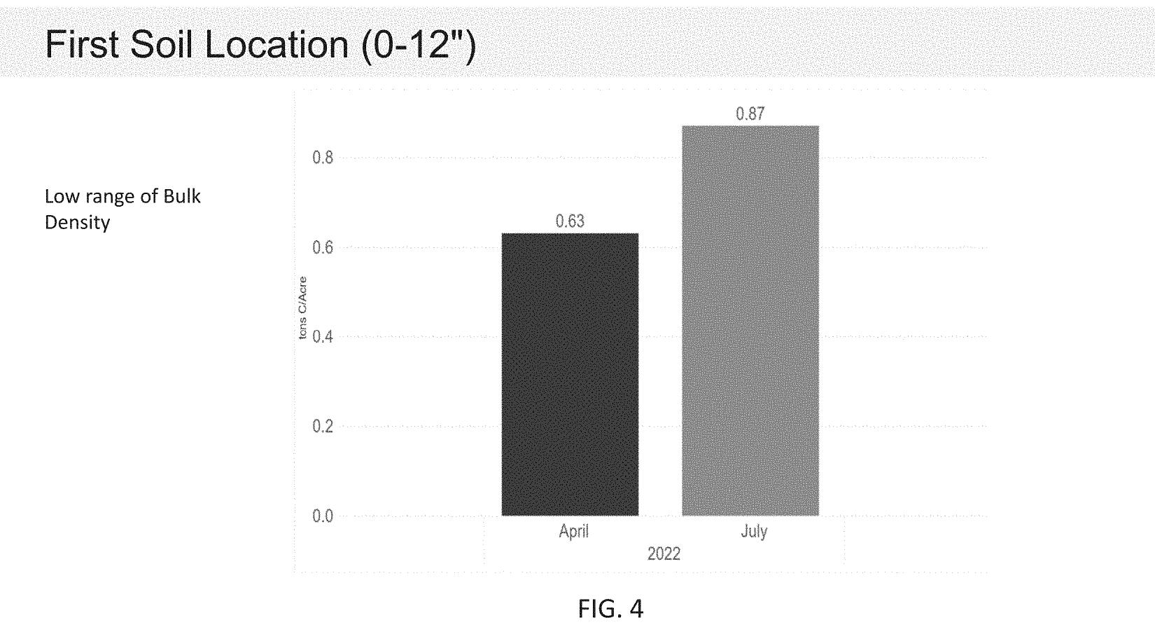

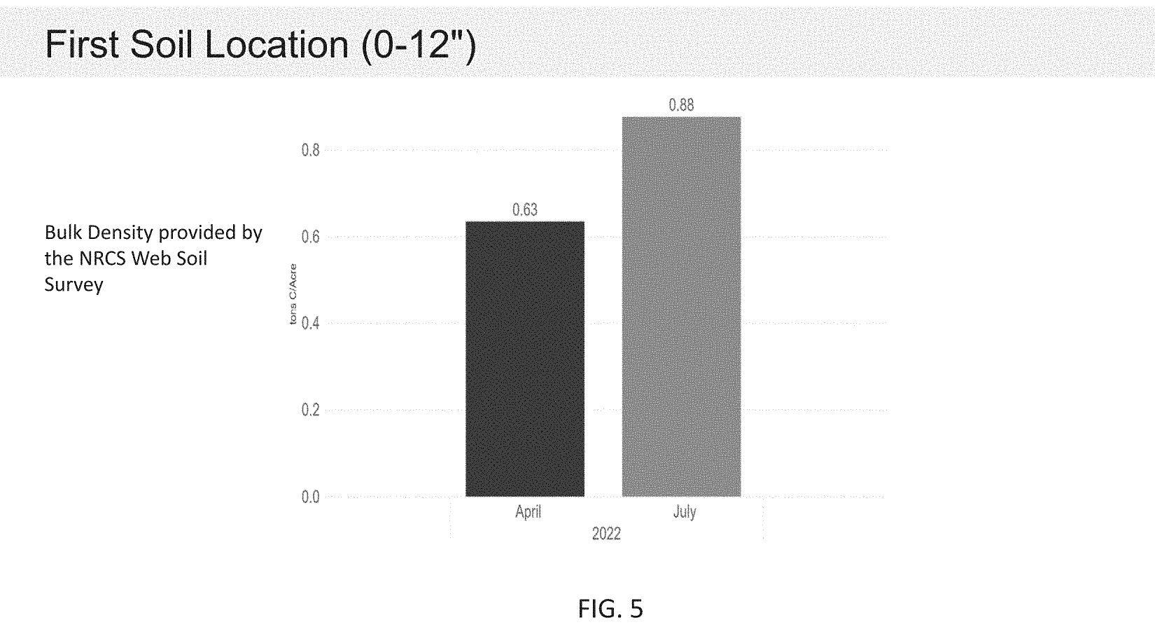

FIG. 1 shows TOC change at soil depths of 0-12″ for a first soil location according to some embodiments. In some embodiments, the first soil location included a potato farm. In some embodiments, the soil was sampled before algae addition in April 2021 at the beginning of a first location cropping cycle, where follow up samples were taken in July 2021 days before harvest according to some embodiments. FIG. 2 illustrates the approximate 38% increase in total organic carbon (TOC) in the soil according to some embodiments. FIG. 3 shows a high range of bulk density according to some embodiments. FIG. 4 shows a low range of bulk density increase for the first location according to some embodiments. FIG. 5 shows bulk density provided by the NRCS Web Soil Survey according to some embodiments.

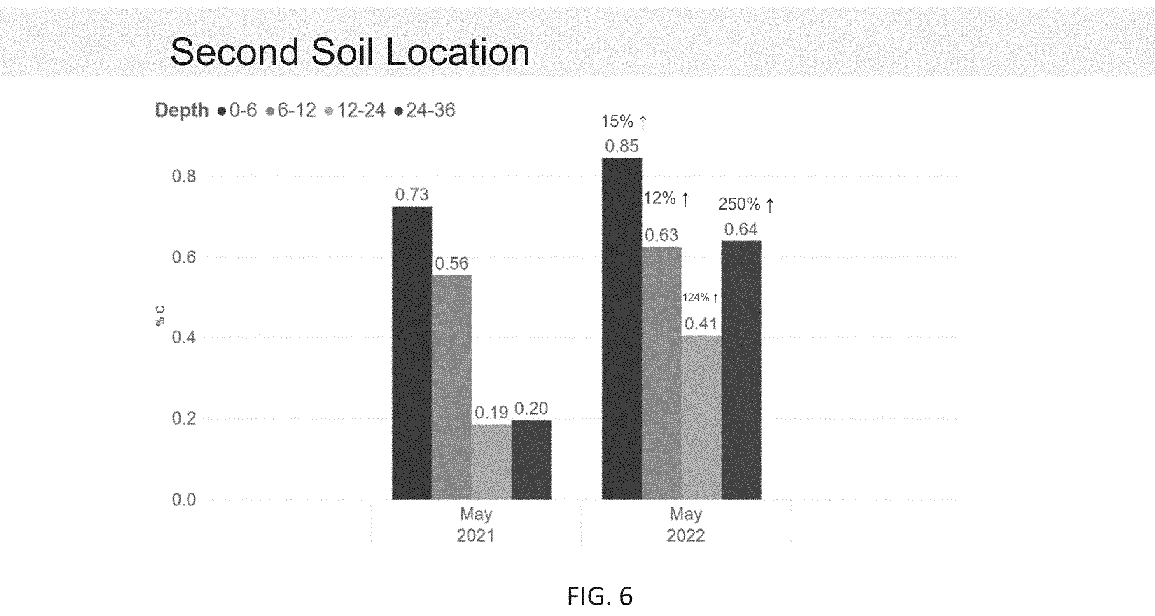

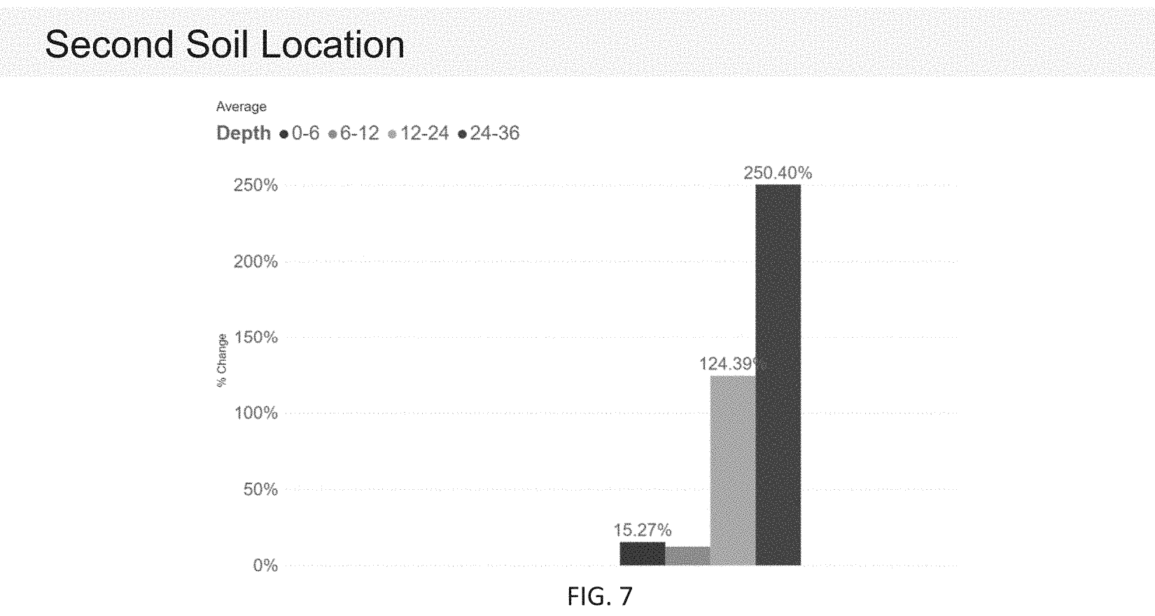

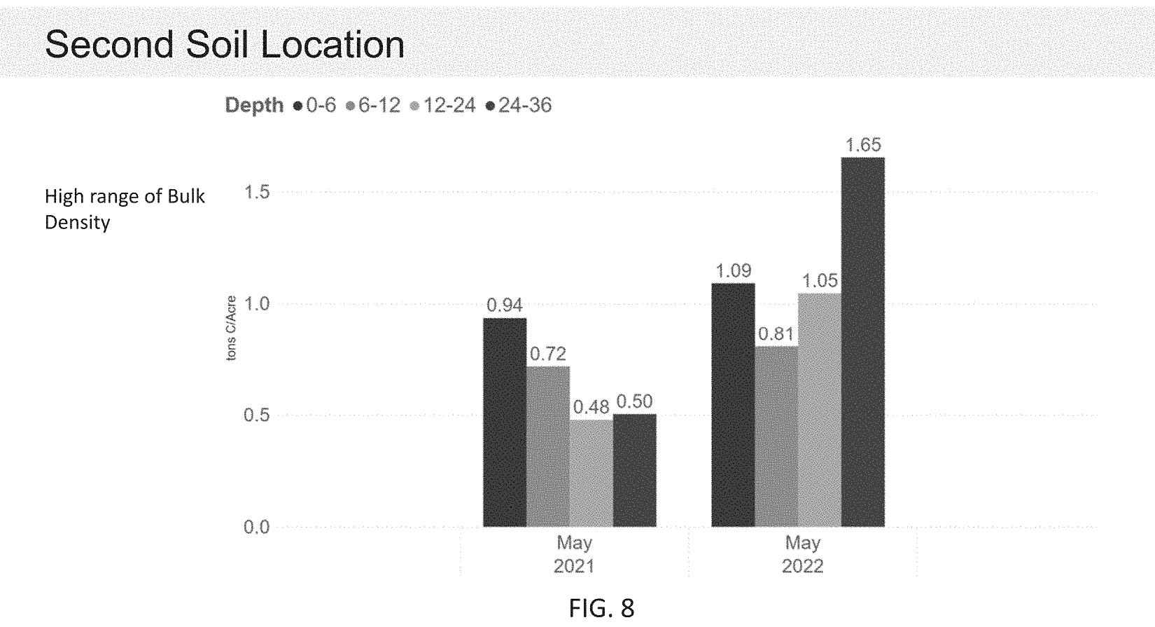

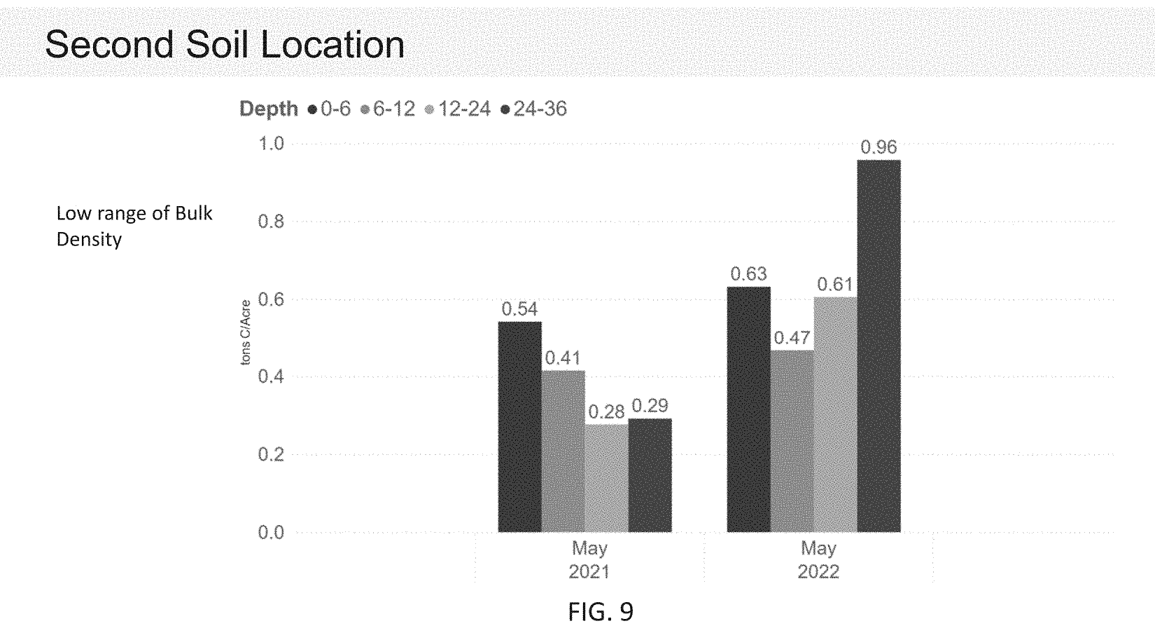

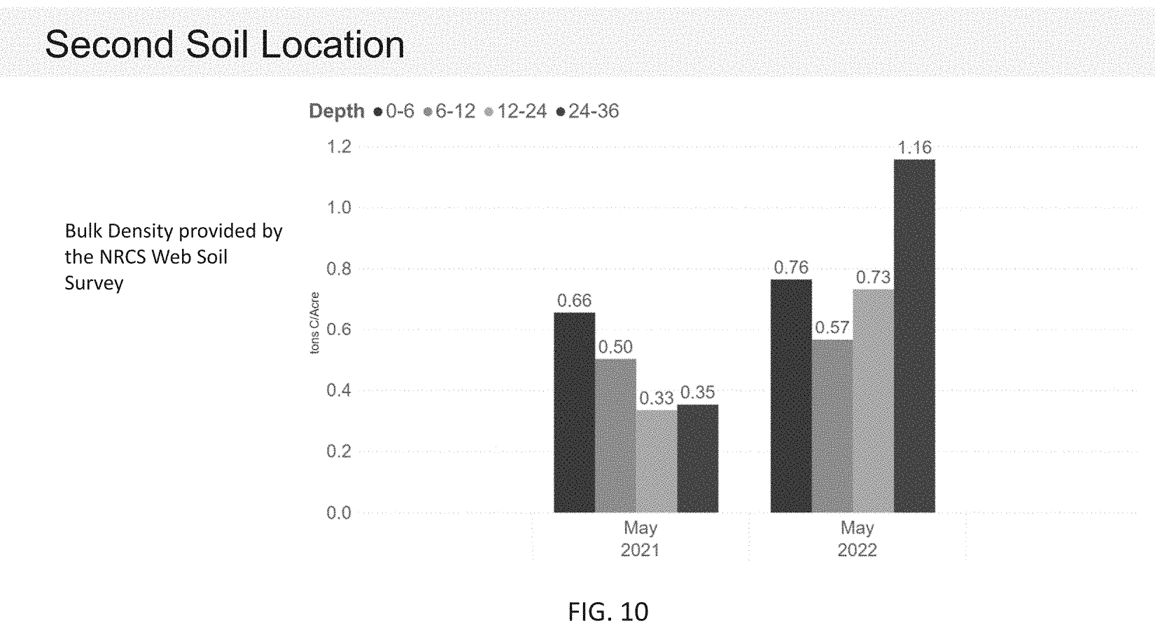

FIG. 6 shows TOC increase at soil depths of 0-6″, 6-12″, 12-24″, and 24-36″ at a second soil location according to some embodiments. In some embodiments, the second soil location included an alfalfa farm according to some embodiments. The data depicts an average of two fields sampled over approximately a year's time according to some embodiments. FIG. 7 illustrates the % change in TOC for the second soil location according to some embodiments. FIG. 8 depicts the change in the high range of bulk density according to some embodiments. FIG. 9 depicts the change in the low range of bulk density according to some embodiments. FIG. 10 shows bulk density provided by the NRCS Web Soil Survey according to some embodiments.

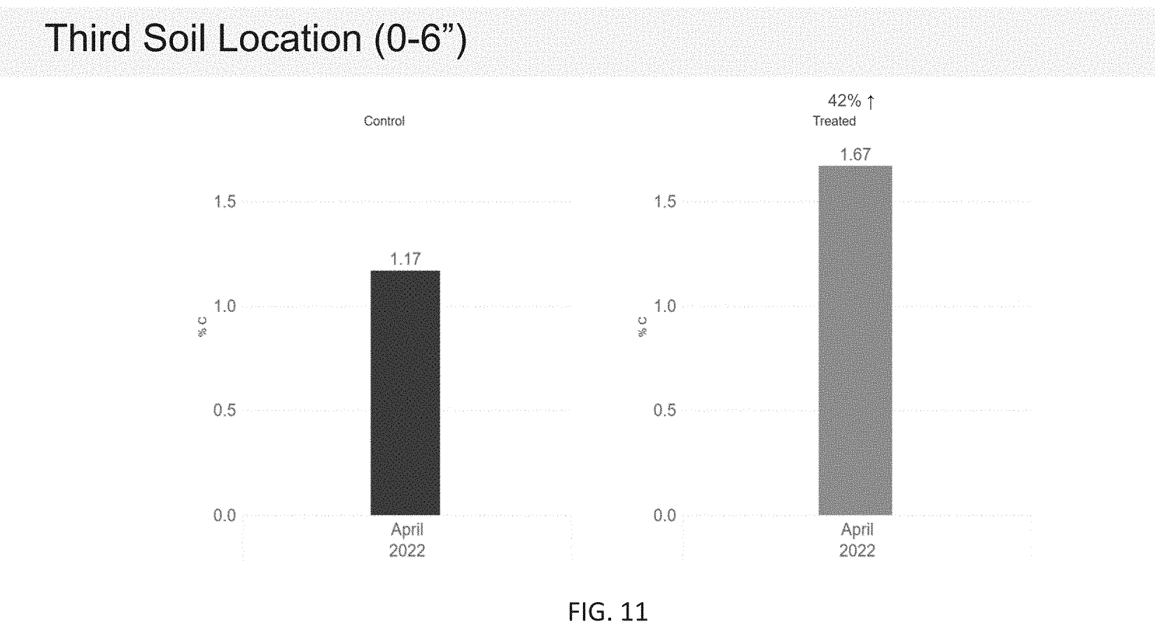

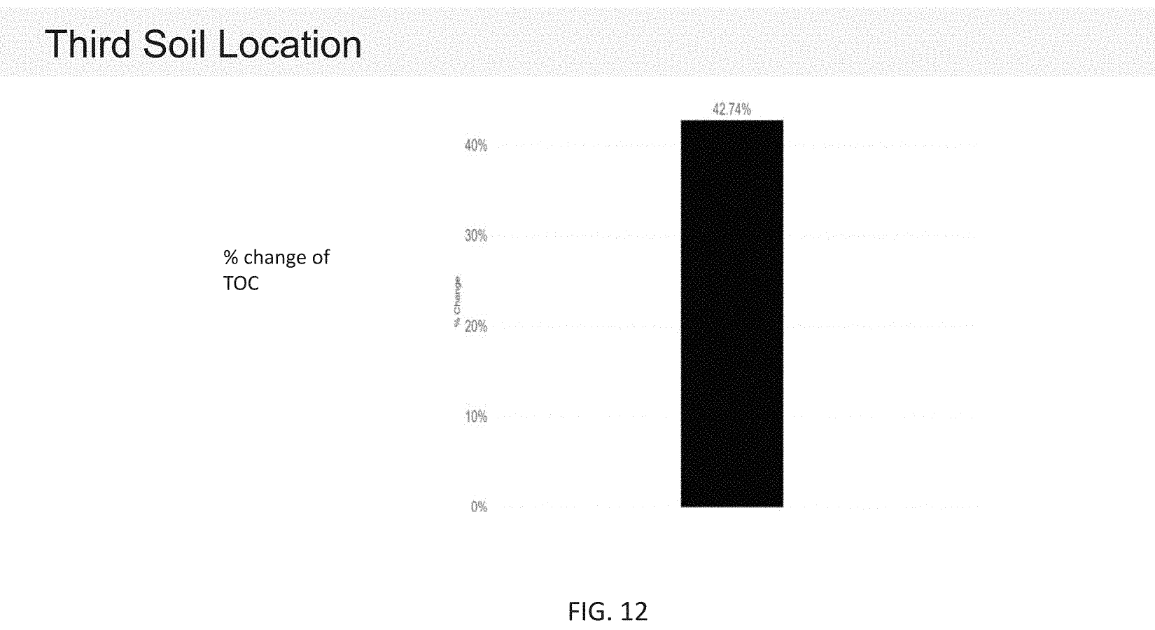

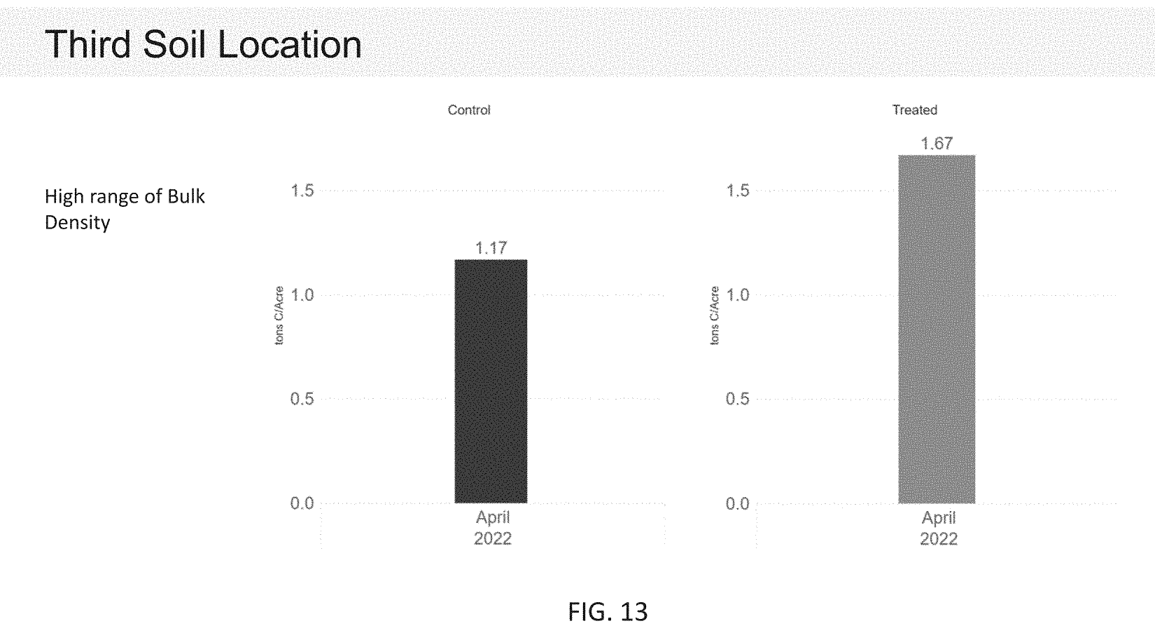

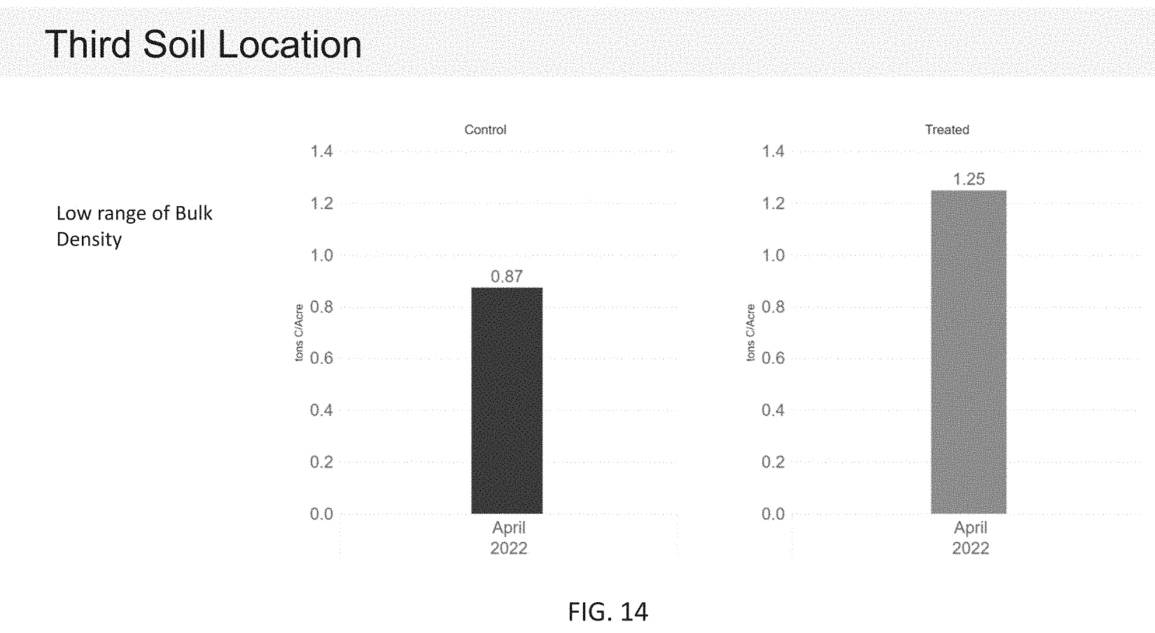

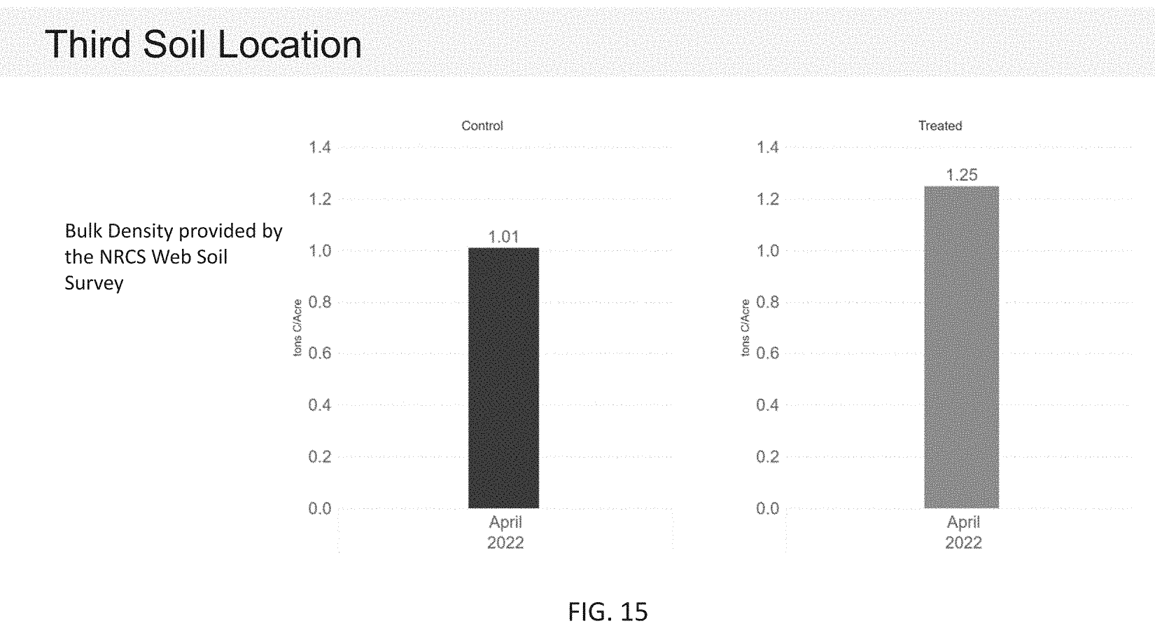

FIG. 11 shows TOC increase at soil depths of 0-6″ at a third soil location according to some embodiments. In some embodiments, the third soil location included a golf course. FIG. 12 shows the % change in TOC according to some embodiments. FIG. 13 depicts the change in the high range of bulk density according to some embodiments. FIG. 14 depicts the change in the low range of bulk density according to some embodiments. FIG. 15 shows bulk density provided by the NRCS Web Soil Survey according to some embodiments.

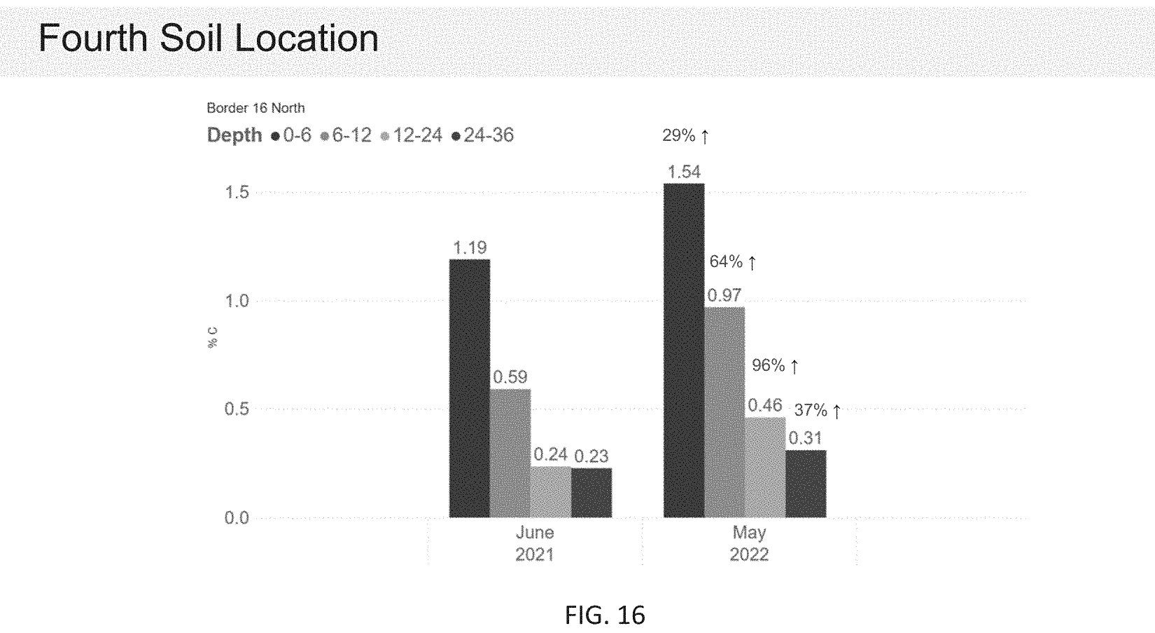

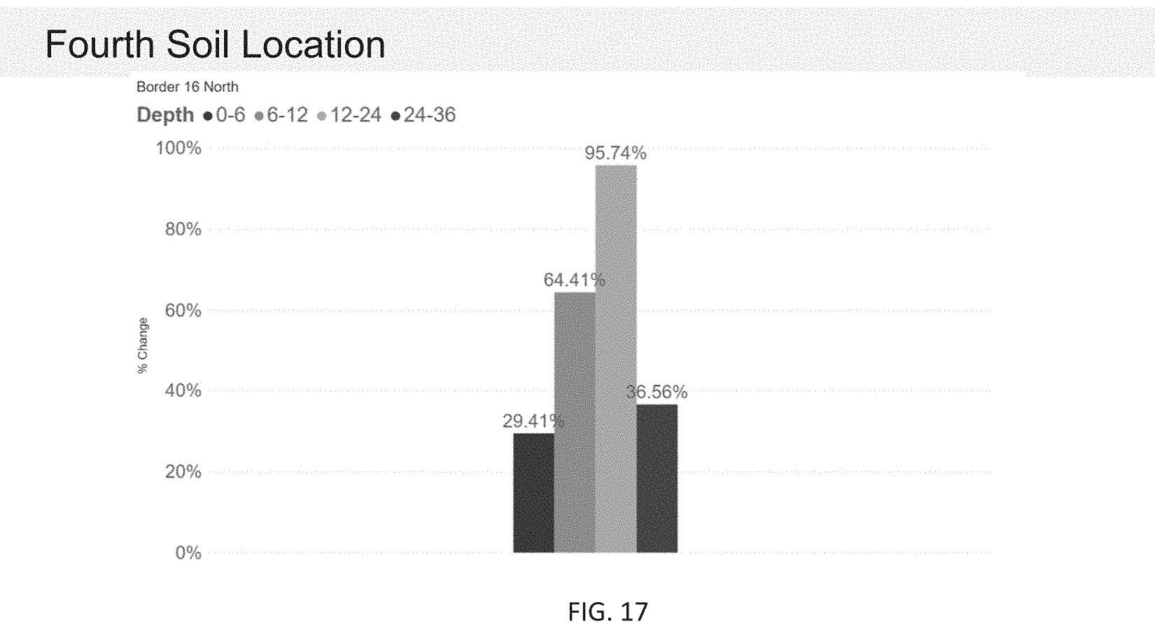

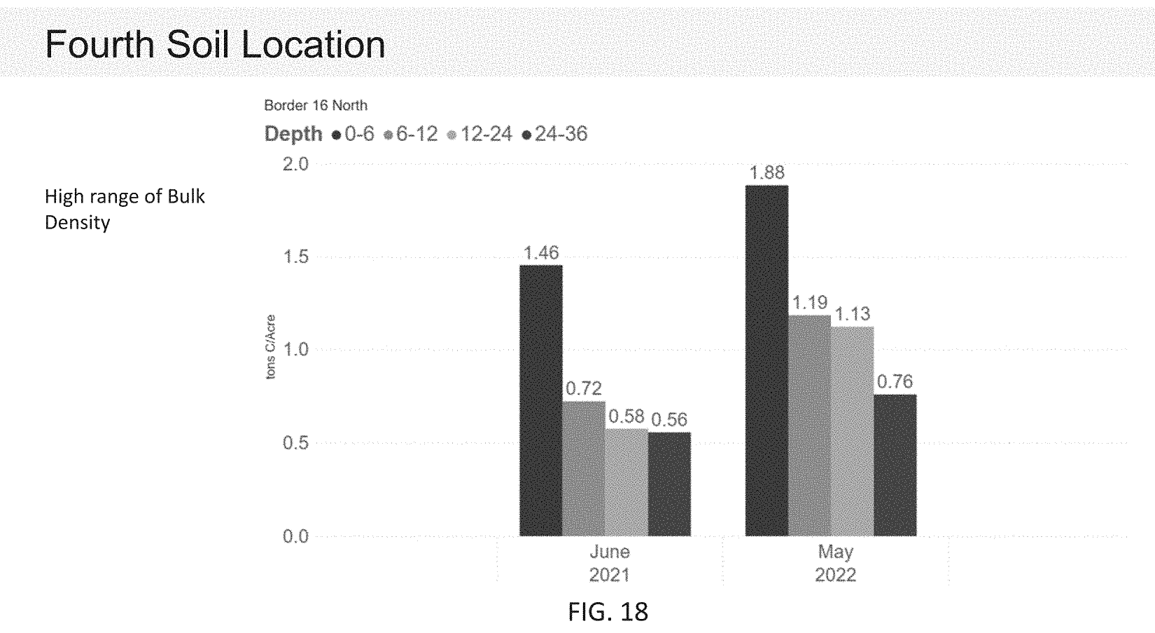

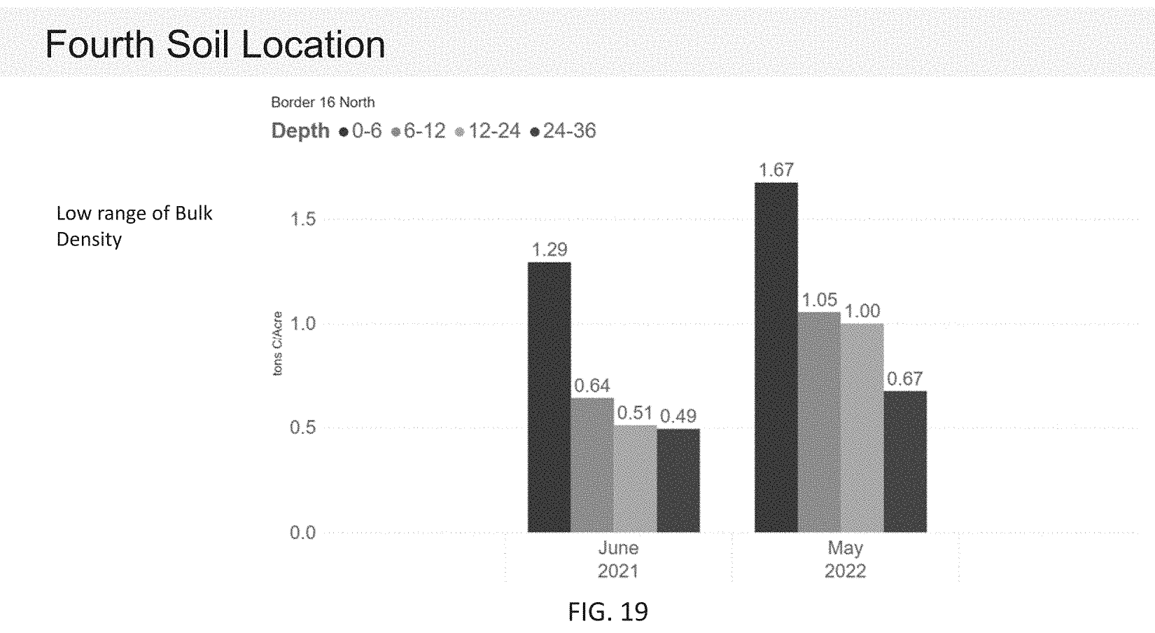

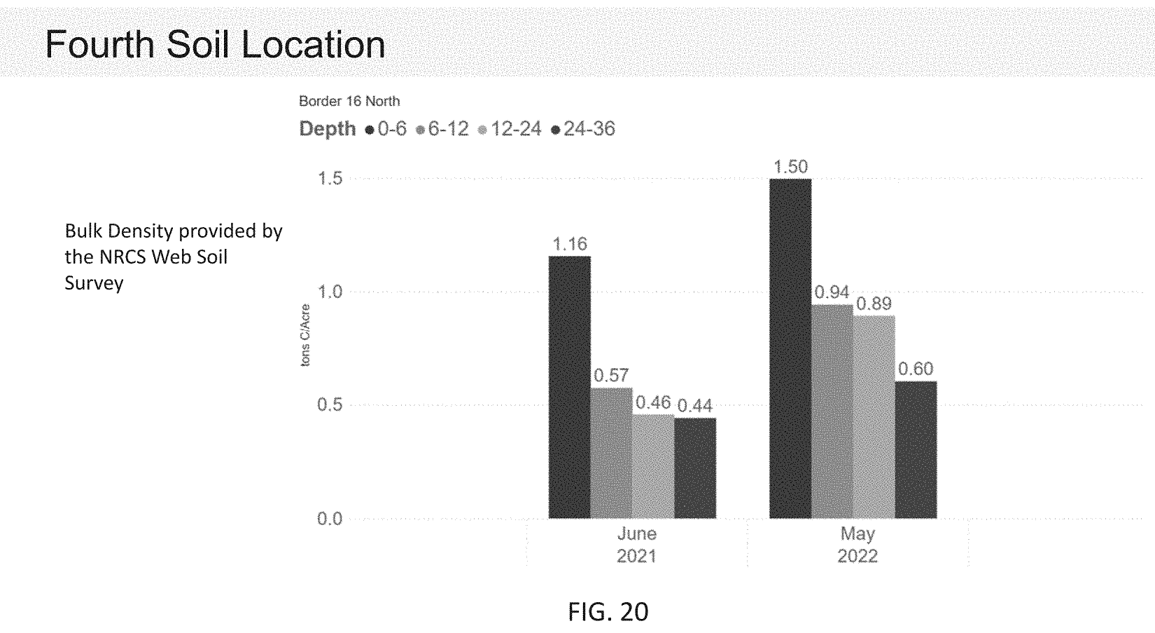

FIG. 16 shows TOC increase at soil depths of 0-6″, 6-12″, 12-24″, and 24-36″ at a fourth soil location according to some embodiments. In some embodiments, the fourth soil location included a farm. FIG. 17 shows the % change in TOC according to some embodiments. FIG. 18 depicts the change in the high range of bulk density according to some embodiments. FIG. 19 depicts the change in the low range of bulk density according to some embodiments. FIG. 20 shows bulk density provided by the NRCS Web Soil Survey according to some embodiments.

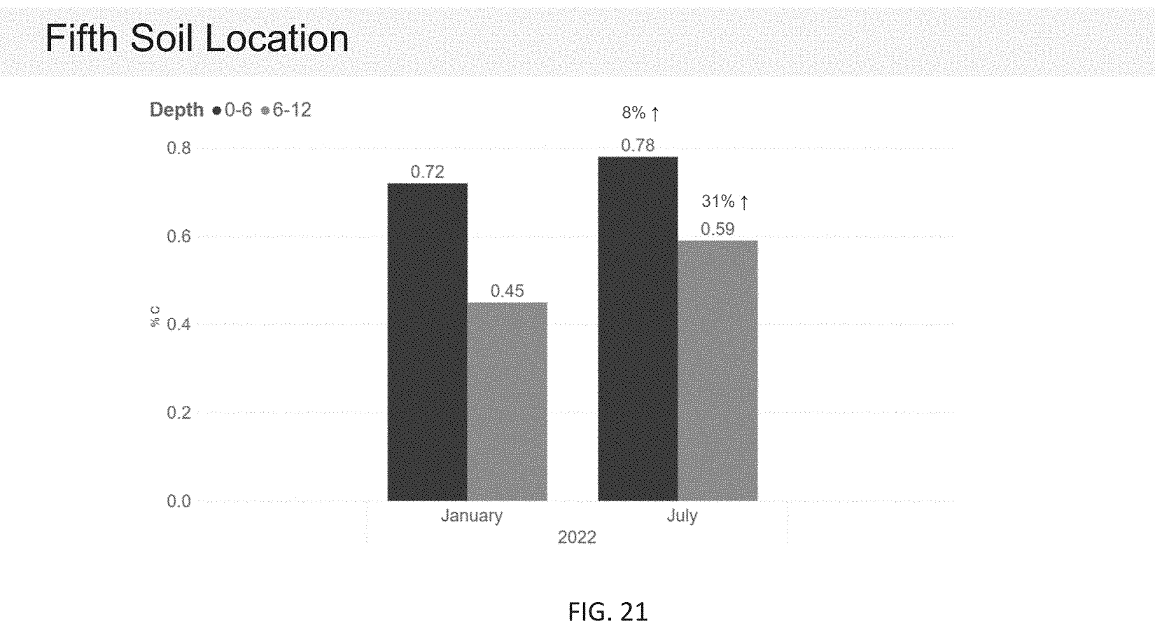

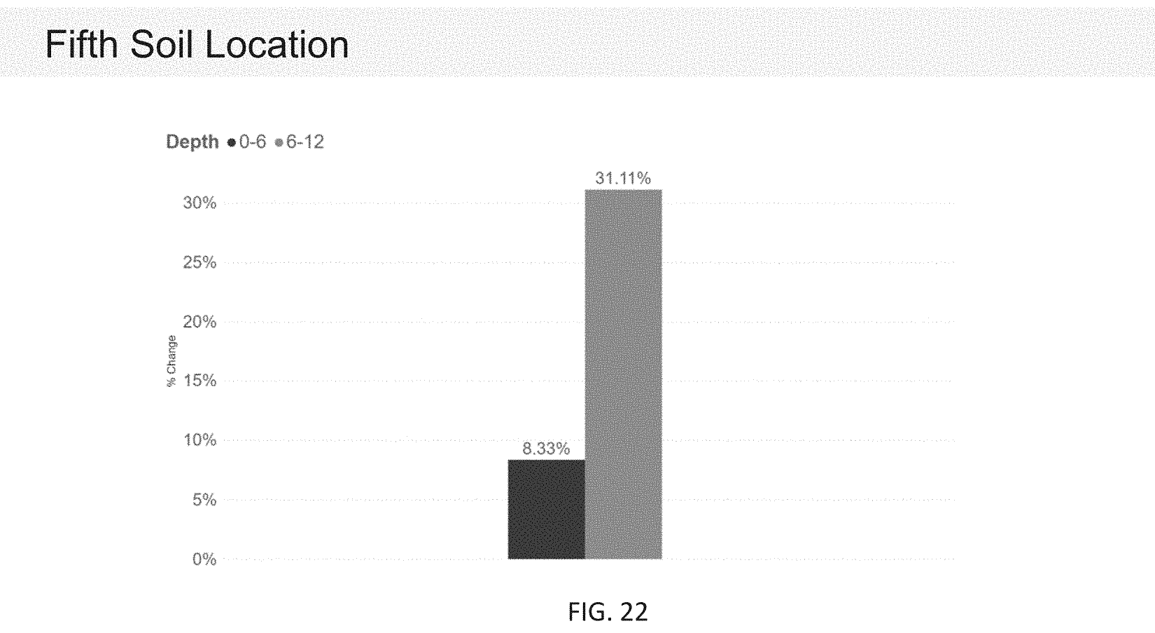

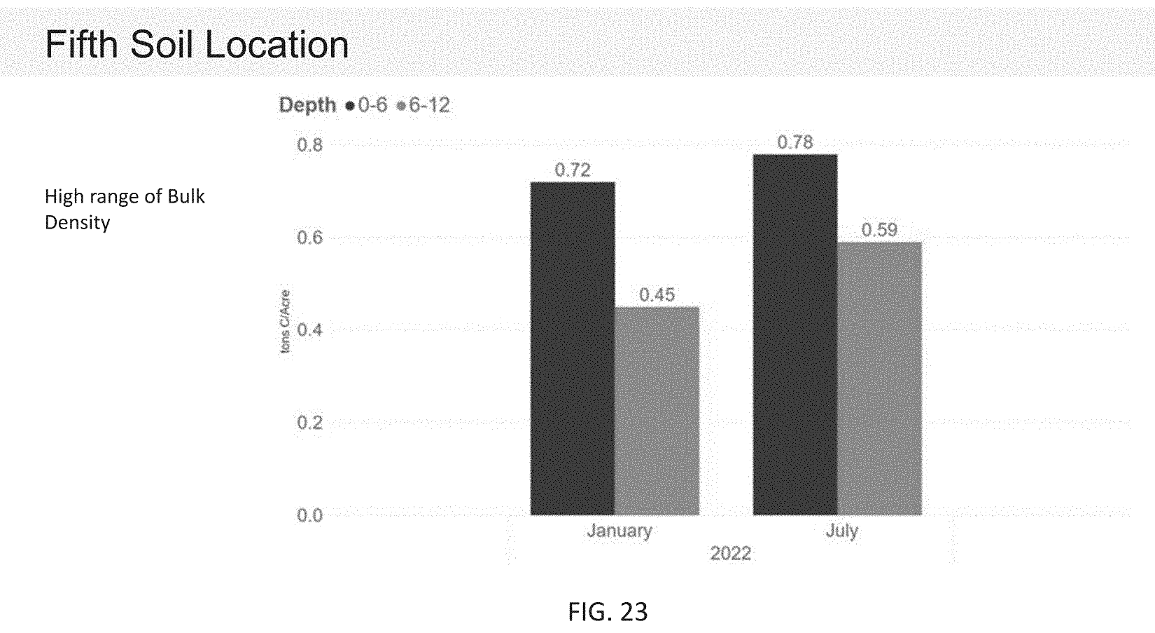

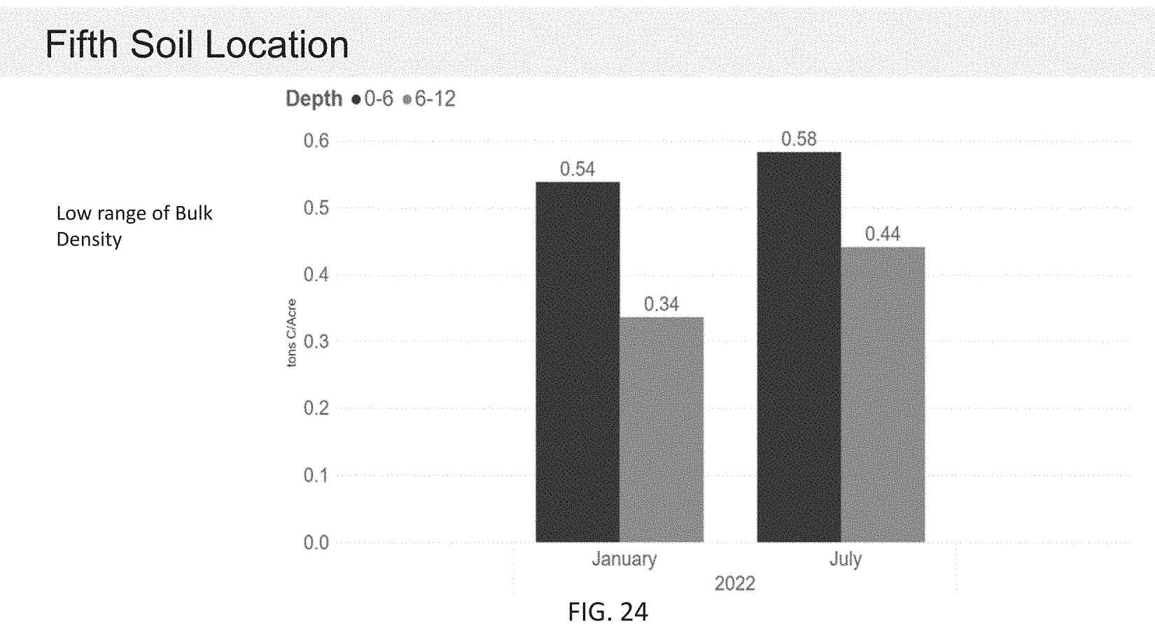

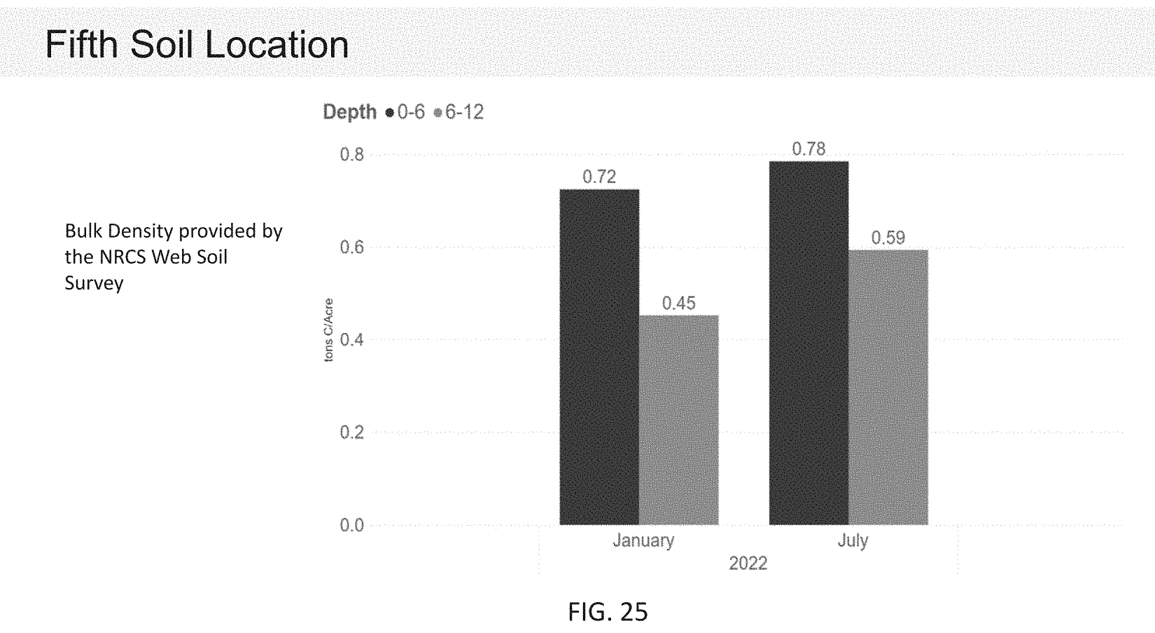

FIG. 21 shows TOC increase at soil depths of 0-6″ and 6-12″ at a fifth soil location according to some embodiments. In some embodiments, the fifth soil location included an almond farm. FIG. 22 shows the % change in TOC according to some embodiments. FIG. 23 depicts the change in the high range of bulk density according to some embodiments. FIG. 24 depicts the change in the low range of bulk density according to some embodiments. FIG. 25 shows bulk density provided by the NRCS Web Soil Survey according to some embodiments.

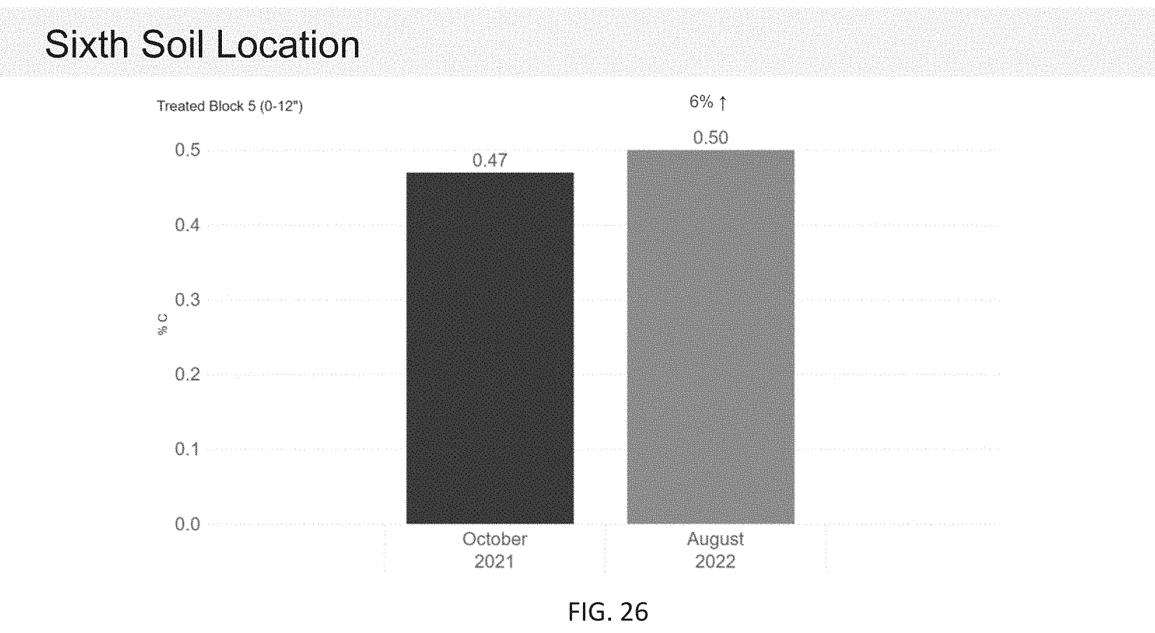

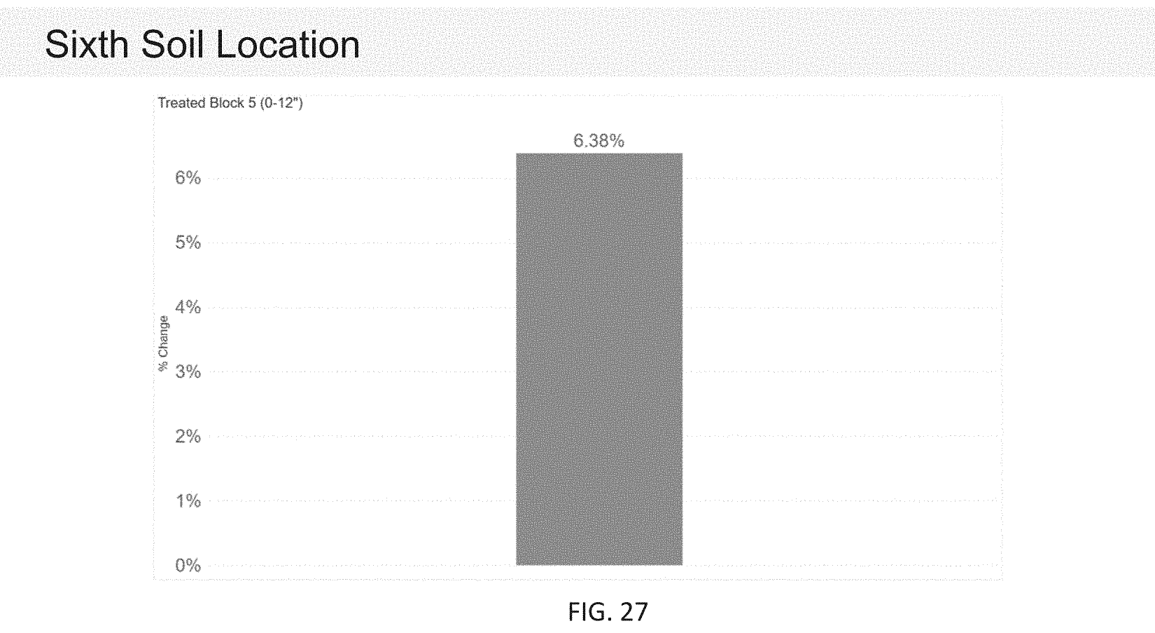

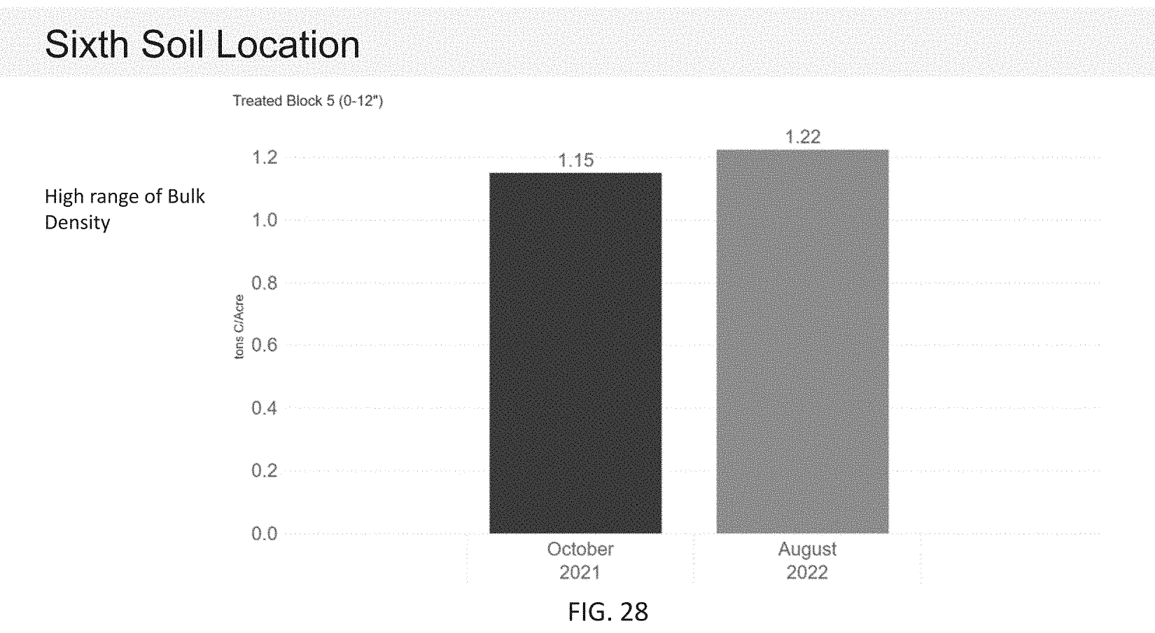

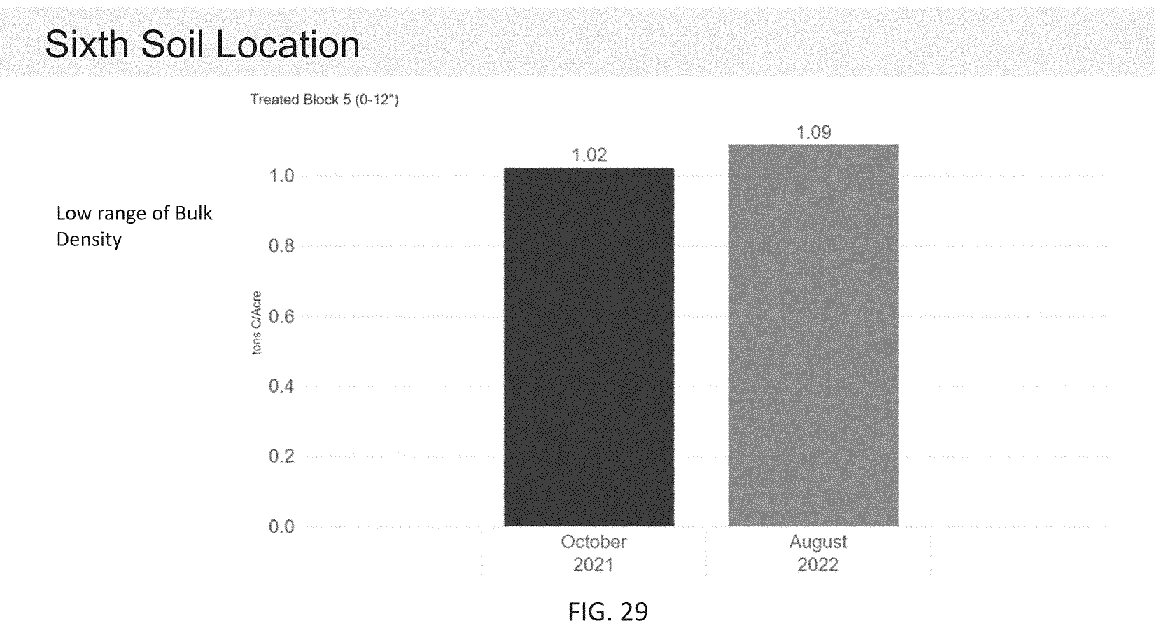

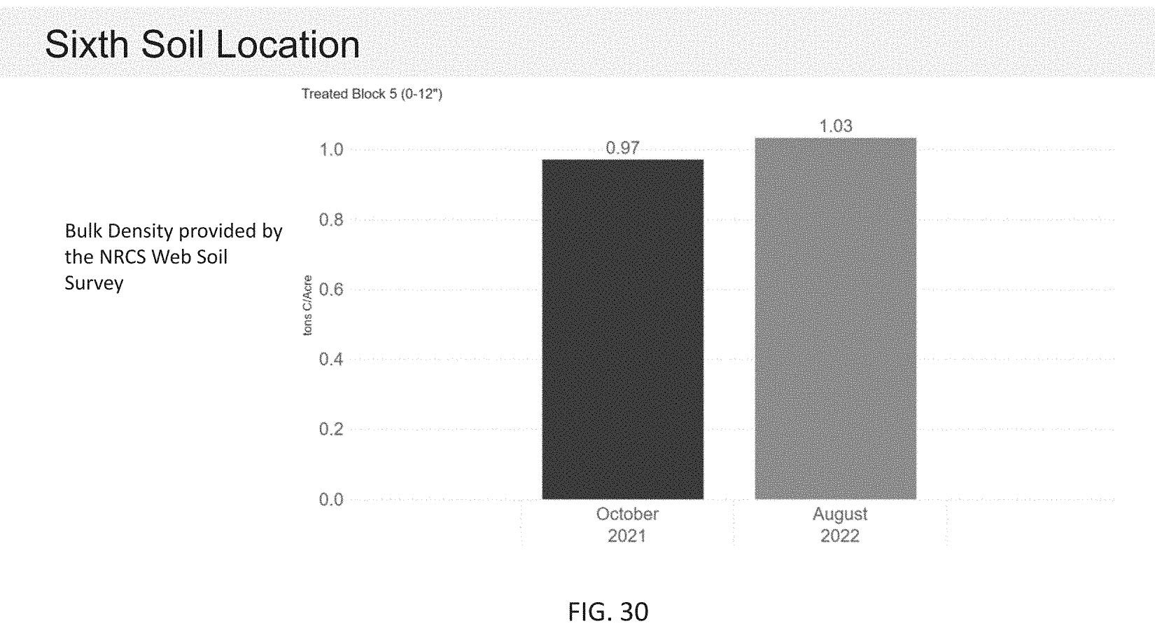

FIG. 26 shows TOC increase at soil depths of 0-12″ at a sixth soil location according to some embodiments. In some embodiments, the sixth soil location included a first section of a strawberry ranch. FIG. 27 shows the % change in TOC according to some embodiments. FIG. 28 depicts the change in the high range of bulk density according to some embodiments. FIG. 29 depicts the change in the low range of bulk density according to some embodiments. FIG. 30 shows bulk density provided by the NRCS Web Soil Survey according to some embodiments.

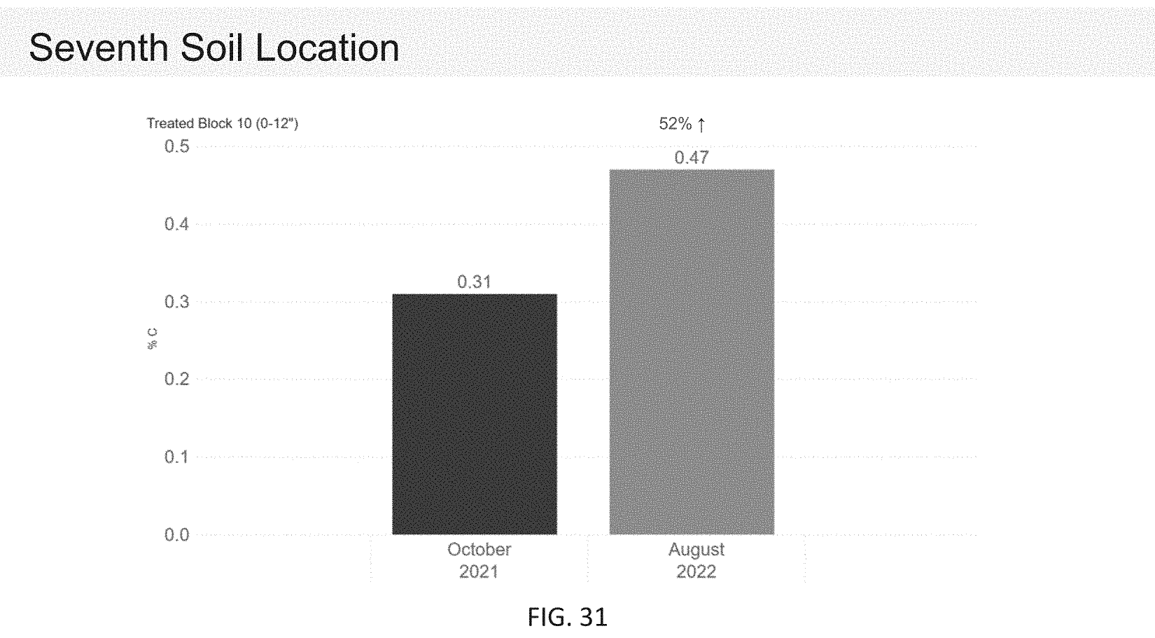

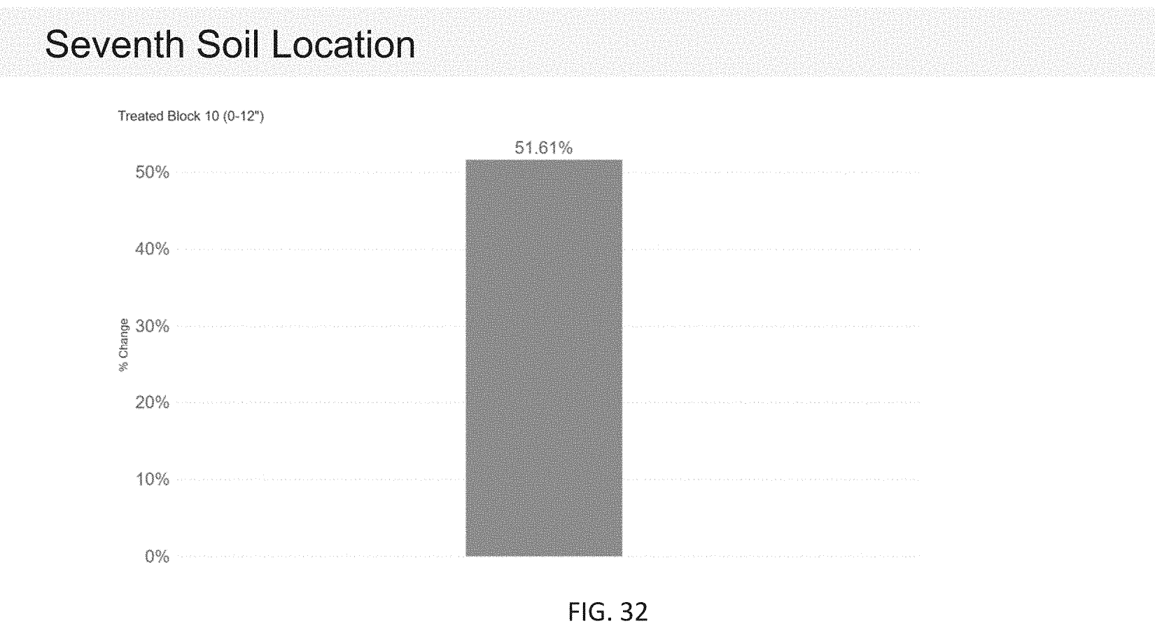

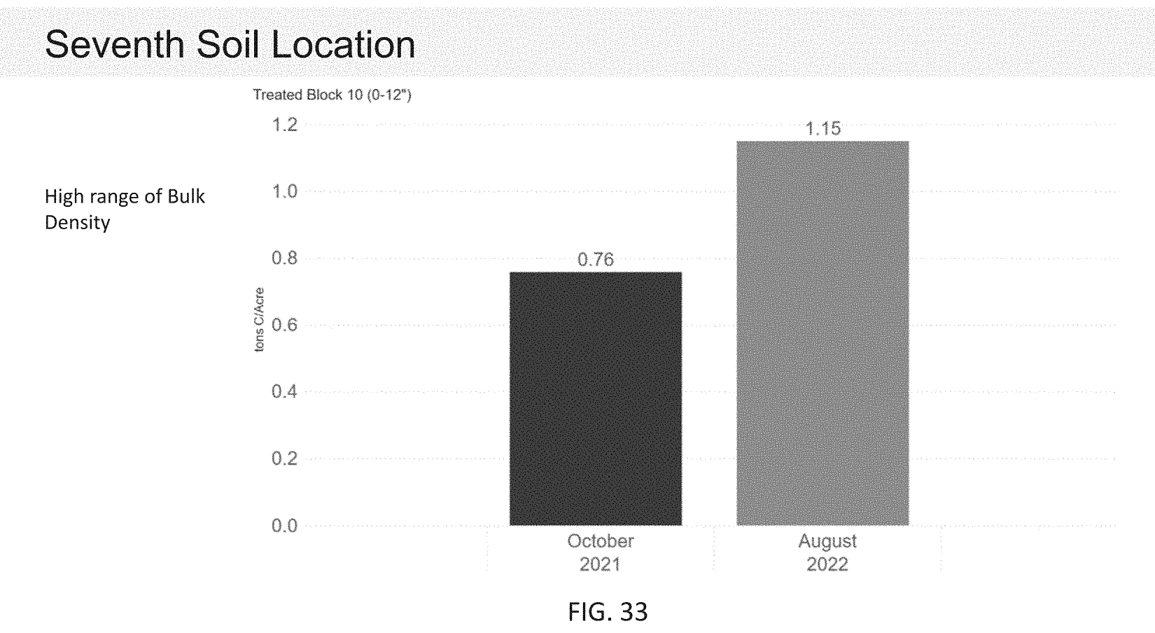

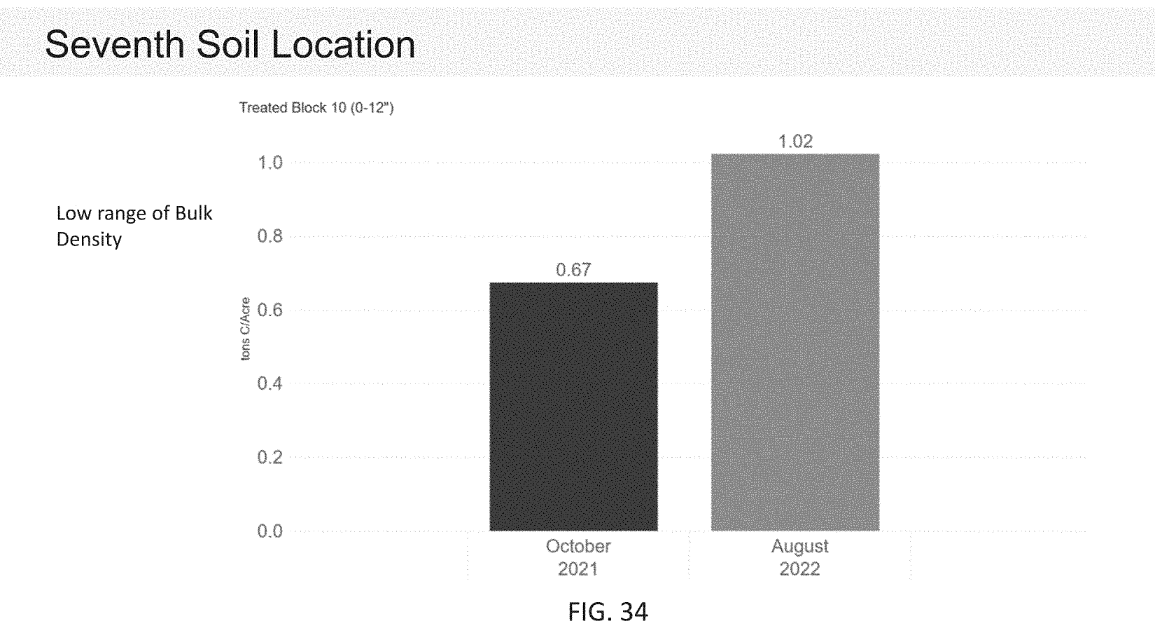

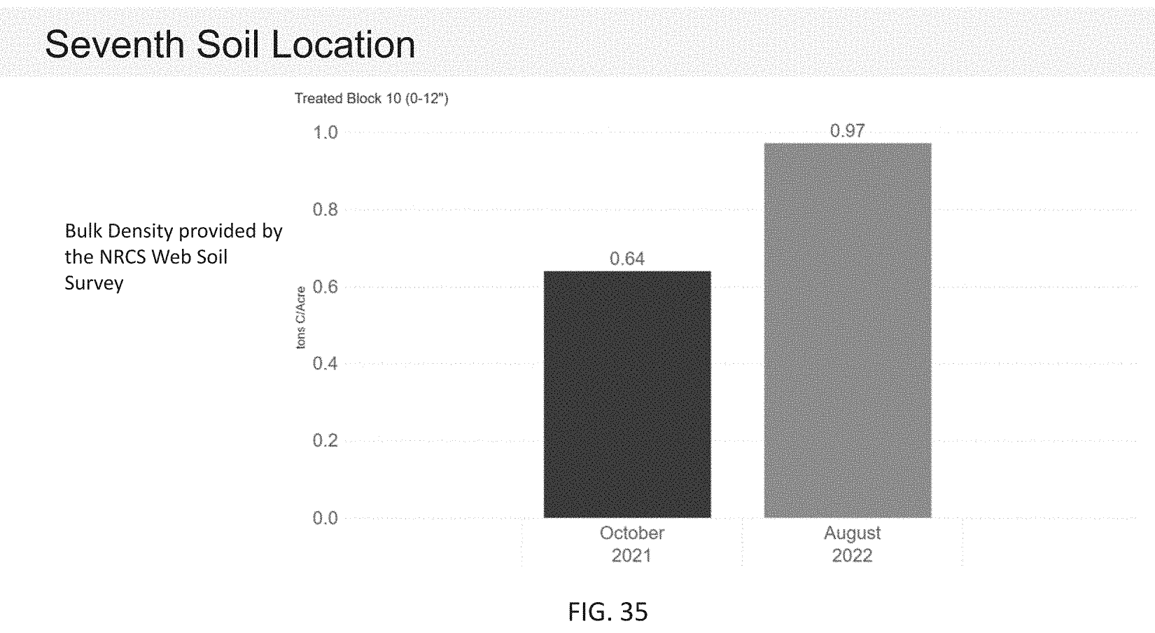

FIG. 31 shows TOC increase at soil depths of 0-12″ at a seventh soil location according to some embodiments. In some embodiments, the seventh soil location included a second section of the same strawberry ranch. FIG. 32 shows the % change in TOC according to some embodiments. FIG. 33 depicts the change in the high range of bulk density according to some embodiments. FIG. 34 depicts the change in the low range of bulk density according to some embodiments. FIG. 35 shows bulk density provided by the NRCS Web Soil Survey according to some embodiments.

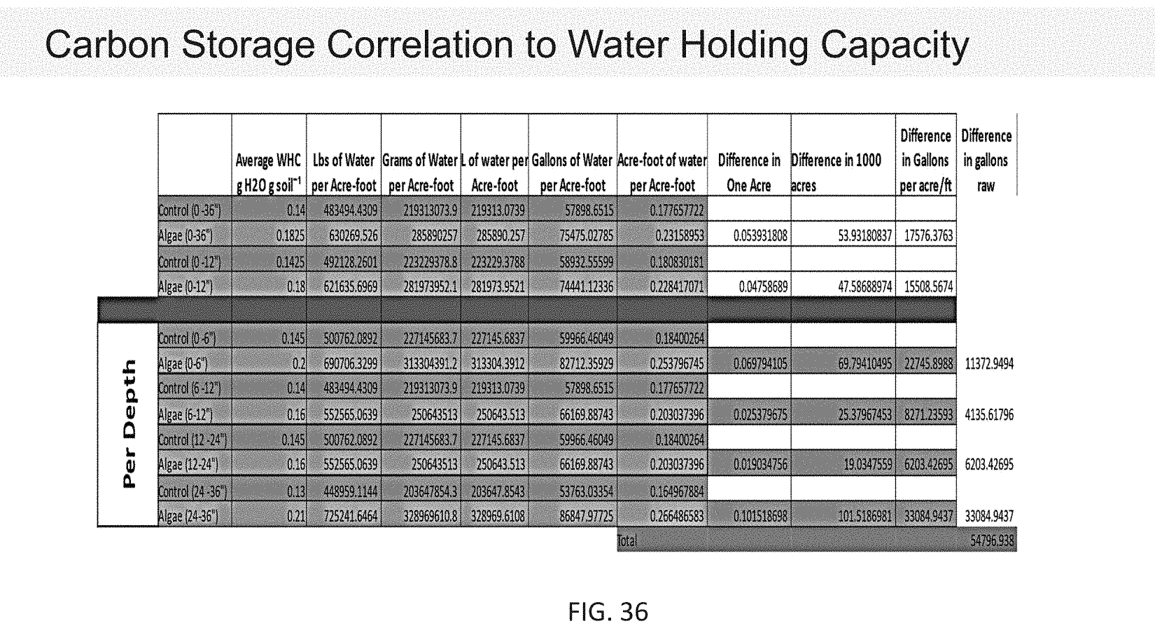

FIG. 36 illustrates an amount of increased water holding capacity for the third soil location as a result of the system and methods described herein according to some embodiments. As shown the soil's ability to hold water at various depths increased to a total of approximately 55 thousand gallons according to some embodiments.

The subject matter described herein are directed to technological improvements to the field of carbon capture using novel methods to increase carbon concentration in soil. Moreover, the claims presented herein do not attempt to tie-up a judicial exception by simply linking it to a technological field. Indeed, the systems and methods described herein were unknown and/or not present in the public domain at the time of filing, and they provide technologic improvements advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Any text in the drawings is part of the system's disclosure and is understood to be readily incorporable into any description of the metes and bounds of the system. Any functional language in the drawings is a reference to the system being configured to perform the recited function, and structures shown or described in the drawings are to be considered as the system comprising the structures recited therein. It is understood that defining the metes and bounds of the system using a description of images in the drawing does not need a corresponding text description in the written specification to fall with the scope of the disclosure.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a particular action or implementation of method steps.

As used herein, “can” or “may” or derivations there of (e.g., the system can deliver a concentration X) are used for descriptive purposes only and is understood to be synonymous and/or interchangeable with “configured to” (e.g., the control is configured to execute instructions X) when defining the metes and bounds of the system. The phrase “configured to” also denotes the step of configuring a structure to execute a function in some embodiments.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. The recitation “configured to” can also be interpreted as synonymous with operatively connected when used in conjunction with physical structures.

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The previous detailed description is to be read with reference to the figures, in which like elements in different figures have like reference numerals. The figures, which are not necessarily to scale, depict some embodiments and are not intended to limit the scope of embodiments of the system.

Any of the operations described herein that form part of the invention are useful machine operations. The invention also relates to a device or an apparatus for performing these operations.

Although method operations are presented in a specific order according to some embodiments, the execution of those steps do not necessarily occur in the order listed unless explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

What is claimed:

1. A method of capturing carbon comprising:
preparing one or more microbe-containing samples from at least one location of a current or planned plant growth area;
preparing at least one cultured sample by culturing microbes from the sample;
selecting at least one target species of microbe from the at least one cultured sample;
propagating the at least one selected target species of microbe to increase the concentration of the at least one target species of microbe in the at least one cultured sample; and
delivering at least a portion of the at least one target species microbe to at least a portion of the at least one location, wherein at least a portion of the at least one target species of microbe being delivered comprises at least one live microbe.

2. The method of claim 1,
wherein the at least one selected target species of microbe includes an algal species.

3. The method of claim 1,
wherein the at least one selected target species of microbe includes a bacterial species.

4. The method of claim 1,
wherein the at least one selected target species of microbe includes a fungal species.

5. The method of claim 1,
wherein the at least one selected target species of microbe includes a mixotrophic capable algal species.

6. The method of claim 1,
wherein the at least one selected target species of microbe is selected based at least in part on its ability to produce biomass.

7. The method of claim 1,
wherein the propagating of the at least one selected target species of microbe is performed in a bioreactor vessel.

8. The method of claim 6,
wherein the bioreactor vessel is an algae production vessel.

9. The method of claim 6,
wherein the bioreactor vessel is a photobioreactor.

10. The method of claim 6,
wherein the bioreactor is located onsite at the at least one location of a current or planned plant growth area.

11. The method of claim 6,
wherein the bioreactor is located offsite from the at least one location of a current or planned plant growth area.

12. The method of claim 1,
wherein a result of the delivering at least a portion of the at least one target species of microbe to at least a portion of the at least one location includes an increase in a concentration of the at least one target species in the at least one location.

13. The method of claim 10,
wherein the increase in concentration comprises an increase in concentration beyond a naturally occurring concentration of the at least one target species of microbe of the portion of the at least one location.

14. The method of claim 10,
wherein the increase in concentration leads to a reduction in soil salinity.

15. The method of claim 10,
wherein the increase in concentration leads to an increase in soil organic carbon.

16. The method of claim 10,
wherein the increase in concentration leads to an increase in soil organic matter (SOM).

17. The method of claim 10,
wherein the increase in concentration leads to an increase in the bioavailability of macro and micronutrients.

18. The method of claim 10,
wherein the increase in concentration leads to improved soil permeability and water retention.

19. The method of claim 10,
wherein the increase in concentration leads to improved crop yields in at least a portion of the at least one location.

20. The method of claim 10,
wherein the increase in concentration leads to improved nutrient value and/or overall quality of one or more crops grown in at least a portion of the at least one location.
